(19) United States
(12) Patent Application Publication
(10) Pub. No.: US 2013/0231290 A1
(43) Pub. Date: Sep. 5, 2013

(54) METHODS OF DIAGNOSING AND TREATING NEURODEGENERATIVE DISEASES

(71) Applicant: Jie Wu, Avondale, AZ (US)

(72) Inventor: Jie Wu, Avondale, AZ (US)

(73) Assignee: DIGNITY HEALTH, Phoenix, AZ (US)

(21) Appl. No.: 13/885,534

(22) PCT Filed: Nov. 18, 2011
(86) PCT No.: PCT/US11/61541

§ 371 Date: May 15, 2013

Related U.S. Application Data
(60) Provisional application No. 61/415,291, filed on Nov. 18, 2010.

(51) Int. Cl. A61K 38/17; A61K 31/13; G01N 33/68; A61K 31/439; A61K 31/47; C12Q 1/68

(52) U.S. Cl. CPC A61K 38/1703; A61K 31/47; C12Q 1/6883; G01N 33/6896; A61K 31/439; A61K 31/13
USPC 514/17.8; 514/312; 435/6.11; 435/7.21; 514/279; 514/17.7; 514/661

(57) ABSTRACT

The present invention relates to methods of diagnosing, treating and prognosing mental disorders, such as Alzheimer's Disease. In one embodiment, the present invention provides a method of treating Alzheimer's Disease by inhibiting dysfunctional signaling of α7 nAChRs in the medial septum region of an individual.

This application claims priority to U.S. Ser. No. 61/415,291 filed Nov. 18, 2010, the contents of all of which are herein incorporated by reference.

### FIELD OF THE INVENTION

The present invention relates to methods and compositions related to nicotinic acetylcholine receptors as related to neurodegenerative diseases and/ox conditions.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Nicotinic acetylcholine receptors (nAChRs) in mammals exist as a diverse family of channels composed of different, pentameric combinations of subunits derived from at least sixteen genes (Lukas et al., 1999; Jensen et al., 2005). Functional nAChRs can be assembled as either heteromers containing α and β subunits or as homomers containing only α subunits (Lukas et al., 1999; Jensen et al., 2005). In the mammalian brain, the most abundant forms of nAChRs are heteromeric α4β2-nAChRs and homomeric α7-nAChRs (Whiting et al., 1987; Flores et al., 1992; Gopalakrishnan et al., 1996; Lindstrom, 1996; Lindstrom et al., 1996). α7-nAChRs appear to play roles in the development, differentiation, and pathophysiology of the nervous system (Liu et al., 2007b; Mudo et al., 2007).

nAChRs have been implicated in Alzheimer's disease (AD), in part because significant losses in radioligand binding sites corresponding to nAChRs have been consistently observed at autopsy in a number of neocortical areas and in the hippocampi of patients with AD (Burghaus et al., 2000; Nordberg, 2001). Attenuation of cholinergic signaling is known to impair memory, and nicotine exposure improves cognitive function in AD patients (Levin and Rezvani, 2002). In addition, several studies have suggested that the activation of α7-nAChR function alleviates amyloid-β (Aβ) toxicity. For instance, stimulation of α7-nAChRs inhibits amyloid plaque formation in vitro and in vivo (Geerts, 2005), activates α-secretase cleavage of amyloid precursor protein (APP) (Lahiri et al., 2002), increases acetylcholine (ACh) release and facilitates Aβ internalization (Nagele et al., 2002), inhibits activity of the MAPK/NF-kB/c-myc signaling pathway (Liu et al., 2007a), and reduces Aβ production and attenuates tau phosphorylation (Sadot et al., 1996). These findings suggest that cholinergic signaling, mediated through α7-nAChRs, not only is involved in cognitive function, but also could protect against a wide variety of insults associated with AD (Sivaprakasam, 2006). Conversely, impairment of α7-nAChR-mediated cholinergic signaling during the early stage(s) of AD might play a pivotal role in AD pathophysiology.

In rat basal forebrain cholinergic neurons, α7 and β2 are the predominant nAChR subunits, and they were found to co-localize (Azam et al., 2003). Thus far, there has been no evidence that α7 and β2 subunits co-assemble to form functional nAChRs naturally, although functional α7β2-nAChRs have been reported using a heterologous expression system (Khiroug et al., 2002). As described herein, however, the inventors demonstrate that heteromeric α7β2-nAChRs exist in rodent basal forebrain cholinergic neurons and have high sensitivity to Aβ. There is a need in the art for a greater understanding of the role of nAChRs in learning and memory disorders, specifically Alzheimer's Disease, both in their functional characterization as well as the development of novel treatments for Alzheimer's Disease.

Particularly, which targets specifically mediate Aβ toxicity still remains elusive. There is growing evidence that α7 type nAChRs are important in AD pathogenesis and therapy, based on reports that the activation of α7-nAChRs significantly enhances cognitive function (Levin and Rezvani, 2002; Leiser et al., 2009). This has lead to the use of α7-nAChR agonists to treat AD 4-7 because enhancing α7-nAChR function is supposed to improve AD learn and memory deficits (Bencherif and Schmitt, 2002; Buccafusco et al., 2005; Buckingham et al., 2009; D'Andrea and Nagele, 2006). However, several recent clinical trials for therapies using α7-nAChR agonists have failed (Biton et al., 2007; Lopez-Hernandez et al., 2007; Taly et al., 2009). And in fact, high levels of α7-nAChRs of mRNA and protein are expressed in both AD patients and AD model animals (Jones et al., 2006; Counts et al., 2007b; Hellstron-Lindhal 2004a; Hellstron-Lindhal 2004b; Hellstron-Lindhal 1999; Dinley et al., 2002; Chu et al., 2005; Teaktong et al., 2004). Functionally, α7-nAChR-mediated currents exhibit no impairment in adult (7-month-old) APP transgenic AD mice compared to age-matched wild-type mice (Spencer et al., 2006). In addition, recent data shows that an α7-nAChR agonist (4-OH-GTS-21) actually protects deficient cholinergic function in wild type (WT), but not in APP transgenic AD mice (Ren et al., 2007). Even this α7-nAChR agonist drug nonetheless reduces cholinergic cell size in the more heavily amyloid-depositing APP/PS1 mice (Ren et al., 2007). Together, this suggests that in both AD model animals and AD patients, α7-nAChRs likely exhibit hyper-rather than hypo-expression and function in hippocampal neurons. There is a need to understand whether α7-nAChRs mediates AD pathogenesis and if antagonism of α7-nAChRs is a potential strategy for AD therapy (Dziewczapolski et al., 2009)

### SUMMARY OF THE INVENTION

The following embodiments and aspects thereof are described and illustrated in conjunction with compositions and methods which are meant to be exemplary and illustrative, not limiting in scope. In one embodiment, the invention includes a method of treating a neurodegenerative disorder in an individual, including providing a composition capable of inhibiting dysfunctional signaling of α7 nicotinic acetylcholine receptors (nAChRs), and administering a therapeutically effective amount of the composition to inhibit dysfunctional signaling of α7 nAChRs to treat the neurodegenerative disorder. In another embodiment, the α7 nAChRs are heteromeric α7β2 nAChRs. In another embodiment the composition capable of inhibiting dysfunctional signaling of α7 nAChRs is an β2 nAChR antagonist. In another embodiment, the composition capable of inhibiting dysfunctional signaling of α7 nAChRs is an α7 nAChR antagonist. In another embodiment, the neurodegenerative disorder is Alzheimer's Disease, dementia, Parkinson's Disease and/or epilepsy. In another embodiment, the neurodegenerative disorder is an early stage form of Alzheimer's Disease. In another embodiment, the composition capable of inhibiting dysfunctional signaling of α7 nAChRs comprises a compound includes kynurenic acid (KYNA), methyllycaconitine (MLA), α-bungarotoxin (BGT), cholinesterase inhibitor, memantine, and/or α-conotoxin, or a pharmaceutical equivalent, derivative, analog and/or salt thereof. In another embodiment, inhibiting the dysfunctional signaling of α7 nAChRs includes restoring function of α7β2 nAChRs. In another embodiment, inhibiting the dysfunctional signaling of α7 nAChRs includes protecting α7β2 nAChRs from amyloid 3 (Aβ) effects. In another embodiment, inhibiting the dysfunctional signaling of α7 nAChRs includes a reduction in neuronal hyperexcitation. In another embodiment, the individual is a human. In another embodiment, the individual is a rodent. In another embodiment, the dysfunctional signaling of α7 nAChRs occurs in the brain medial septum and/or diagonal band in the individual. In another embodiment, the dysfunctional signaling of α7 nAChRs occurs in the hippocampus in the individual.

Another embodiment of the invention also provides a method of diagnosing a neurodegenerative disorder in an individual, including obtaining a sample from the individual, assaying the sample to determine the presence or absence of dysfunctional signaling of α7 nicotinic acetylcholine receptors (nAChRs) in the individual, and diagnosing the neurodegenerative disorder based on the presence of dysfunctional signaling of α7 nAChRs in the individual. In another embodiment, the α7 nAChRs are heteromeric α7β2 nAChRs. In another embodiment, the individual is a human. In another embodiment, the individual is a rodent. In another embodiment, the neurodegenerative disorder is Alzheimer's Disease, dementia, Parkinson's Disease and/or epilepsy. In another embodiment, the dysfunctional signaling of α7 nAChRs occurs in the brain medial septum and/or diagonal band in the individual. In another embodiment, the dysfunctional signaling of α7 nAChRs occurs in the hippocampus in the individual. In another embodiment, the neurodegenerative disorder is non-responsive to treatment with galantamine, or a pharmaceutical equivalent, derivative, analog and/or salt thereof. In another embodiment, prior to obtaining the sample the individual is suspected of having a neurodegenerative disorder. In another embodiment, prior to obtaining the sample the individual demonstrates susceptibility to seizures. In another embodiment, prior to obtaining the sample the individual demonstrates abnormal θ oscillations.

Another embodiment of the invention also provides a method of prognosing the onset of Alzheimer's Disease and/or dementia in an individual, including obtaining a sample from the individual, assaying the sample to determine the presence or absence of dysfunctional signaling of α7 nicotinic acetylcholine receptors (nAChRs) in the individual, and prognosing the onset of Alzheimer's Disease and/or dementia based on the presence of dysfunctional signaling of α7 nAChRs in the individual. In another embodiment, the α7 nAChRs are heteromeric α7β2 nAChRs. In another embodiment, the dysfunctional signaling of α7 nAChRs occurs in the brain medial septum and/or diagonal band in the individual. In another embodiment, the dysfunctional signaling of α7 nAChRs occurs in the hippocampus in the individual.

Another embodiment of the invention also provides a method of diagnosing an increased likelihood of an individual developing a neurodegenerative disorder relative to a normal subject, including obtaining a sample from the individual, assaying the sample to determine the presence or absence of dysfunctional signaling of α7 nicotinic acetylcholine receptors (nAChRs) in the individual, diagnosing an increased likelihood of developing the neurodegenerative disorder relative to the normal subject based on the presence of dysfunctional signaling of α7 nAChRs in the individual. In another embodiment, α7 nAChRs are heteromeric α7β2 nAChRs. In another embodiment, neurodegenerative disorder includes Alzheimer's Disease, dementia, Parkinson's Disease and/or epilepsy. In another embodiment, prior to obtaining the sample the individual is suspected of having a neurodegenerative disorder. In another embodiment, prior to obtaining the sample the individual demonstrates susceptibility to seizures.

Another embodiment of the invention also provides a kit, including a quantity of a composition capable of detecting the presence or absence of dysfunctional signaling and/or expression of α7 nicotinic acetylcholine receptors (nAChRs), and instructions for obtaining a sample from an individual, assaying the sample to determine the presence or absence of dysfunctional signaling and/or expression of nAChRs in the individual, and diagnosing an increased likelihood of developing a neurodegenerative disorder relative to the normal subject based on the presence of dysfunctional signaling and/or expression of α7 nAChRs in the individual. In another embodiment, the α7 nAChRs are heteromeric α7β2 nAChRs. In another embodiment, neurodegenerative disorder includes Alzheimer's Disease, dementia, Parkinson's Disease and/or epilepsy. In another embodiment, the kit is disposable.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the identification of cholinergic neurons dissociated from basal forebrain. A: Phase contrast image of a rat MS/DB brain slice (region confirmed using The Rat Brain in Stereotaxic Coordinates, Paxinos and Watson, 1986). MS/DB neurons (phase-contrast images of dissociated neurons; B) exhibited spontaneous action potential firing (C), insensitivity to muscarine (C), action potential adaptation induced by depolarizing pulses (D), and did not show ‘sag’-like responses to hyperpolarizing pulses (E), suggesting they were cholinergic. F: Dissociated neuron (phase contrast, Ph) labeled with lucifer yellow (LY) showed positive ChAT imnmostaining following patch-clamp recording.

FIG. 2 depicts native nAChR-mediated whole-cell current responses. An identified MS/DB cholinergic neuron (no hyperpolarization-induced current, Iₕ) exhibited α7-nAChR-like current responses to 1 mM ACh and 10 mM choline (sensitive to blockade by 1 nM methyllycaconitine; MLA) but not to 0.1 mM RJR-2403, an agonist selective for α4β2-nAChRs (A), whereas an identified VTA DAergic neuron (evident Iₕ) showed both α7-nAChR-like (i.e., choline and MLA-sensitive components) and α4β2-nAChR-like (i.e., RJR-2403-sensitive component) current responses (summed as in the response to ACh) (B). C: typical traces of 10 mM choline-induced currents in MS/DB and VTA DAergic neurons showing different kinetics for current activation/desensitization with a slower response characteristic of MS/DB neurons. D: statistical comparisons of kinetics of 10 mM choline-induced currents in MS/DB cholinergic and VTA DAergic neurons. ***p<0.001.

FIG. 3 depicts nAChR α7 and β2 subunits are co-expressed, co-localize and co-assemble in rat forebrain MS/DB neurons. RT-PCR products from whole brain, VTA and MS/DB regions (A) corresponding to the indicated nAChR subunits or to the housekeeping gene GAPDH were resolved on an agarose gel calibrated by the flanking 100 bp ladders (heavy band is 500 bp) and visualized using ethidium staining. Note that the representative gel shown for whole brain did not contain a sample for the nAChR α3 subunit RT-PCR product, which typically is similar in intensity to the sample on the gel for the VTA and MS/DB. B: quantification of nAChR subunit mRNA levels for RT-PCR amplification followed by Southern hybridization with ³²P-labeled, nested oligonucleotides normalized to the GAPDH internal control and to levels of each specific mRNA in whole rat brain (ordinate: ±S.E.M.) for the indicated subunits. C: From 15 MS/DB neurons tested, after patch-clamp recordings (Ca: representative whole-cell current trace) the cell content was harvested and single-cell RT-PCR was performed, and the results show that α7 and β2 were the two major nAChR subunits naturally expressed in MS/DB cholinergic neurons (Cb-Cd). Double immunofluorescence labeling of a MS/DB neuron using anti-α7 and anti-β2 subunit antibodies revealed that α7 and β2 subunit proteins co-localized, and similar results were obtained using 31 neurons from 12 rats (D). Protein extracts from rat MS/DB (lane 1) or rat VTA (lane 2) or from MS/DB from nAChR β2 subunit knockout (lane 4) or wild-type mice (lane 5) were immunoprecipitated (IP) with a rabbit anti-α7 antibody (Santa Cruz H302; lanes 1, 2, 4, and 5) or rabbit IgG as a control (lane 3). The eluted proteins from the precipitates were analyzed by immunoblotting (IB) with rat monoclonal anti-β2 subunit antibody mAb270 (upper panel) or rabbit anti-α7 antisera H302 (lower panel). The β2 and α7 bands are indicated by arrows (E). All these data demonstrate that nAChR α7 and β2 nAChR subunits are co-assembled in MS/DB neurons.

FIG. 4 depicts antagonist profiles for MS/DB and VTA nAChRs. Concentration-dependent block by MLA (at the indicated concentrations in nM after pre-exposure for 2 ml and continued exposure during agonist application indicated by open bars) of 10 mM choline-induced (applied as indicated by closed bars) whole-cell currents (representative traces shown) in MS/DB (Aa) and VTA (Ab) neurons was not significantly different (p>0.05, Ac). However, choline-induced currents in MS/DB neurons (Ba) were more sensitive to block by DHβE (at the indicated concentrations in μM after pre-exposure for 2 min and continued exposure during agonist application indicated by open bars) than in VTA neurons (Bb; concentration-response profile shown in Bc).

FIG. 5 depicts effects of 1 nM Aβ₁₋₄₂ on α7β2-nAChRs on MS/DB neurons. Typical whole-cell current traces for responses of MS/DB neurons to 10 mM choline challenge at the indicated times after initial challenge alone show no detectable rundown during repetitive application of agonist (2-s exposure at 2-min intervals; Aa). Choline-induced currents in rat MS/DB neurons were suppressed by 1 nM Aβ₁₋₄₂ (continuously applied for 10 min, but responses to challenges with choline are shown at the indicated times of Aβ exposure; Ab) but not by 1 nM scrambled Aβ₁₋₄₂ (as a control; Ac). Choline-induced currents in VTA neurons were not affected by 1 nM Aβ₁₋₄₂ (Ad). B: Normalized, mean (±SE), peak current responses (ordinate) as a function of time (abscissa, min) during challenges with choline alone (□), in the presence of 1 nM Aβ (▴), or in the presence of control, scrambled Aβ (▾) for the indicated numbers of MS/DB neurons, or during challenges with choline in the presence of 1 nM Aβ for the indicated number of VTA neurons () illustrate that only choline-induced currents in rat MS/DB neurons were sensitive to functional inhibition by Aβ.

FIG. 6 depicts inhibition of choline-induced currents in dissociated MS/DB neurons by Aβ₁₋₄₂ was concentration- and form-dependent. A: Normalized, mean (±SE), peak current responses (ordinate) of the indicated numbers of MS/DN neurons as a function of time (abscissa, min) during challenges with choline in the presence of 1 nM scrambled Aβ (▪) or in the presence of 0.1 nM (), 1 nM (▴) or 10 nM (▾) Aβ show concentration dependence of functional block. B: Normalized responses (ordinate) during challenges with choline in the presence of 1 nM monomeric (▪), oligomeric (▴) or fibrillar () Aβ indicate insensitivity to monomeric Aβ and highest sensitivity to peptide oligomers. *p<0.05, **p<0.01, and ***p<0.001.

FIG. 7 depicts effects of Aβ on heterologously-expressed, homomeric α7- and heteromeric α7β2-nAChRs in Xenopus oocytes. Choline (10 mM, 2-s exposure at 2-min intervals)-induced whole-cell current responses in oocytes injected with rat α7-nAChR subunit cRNA alone (Aa, black trace) or with α7 and β2 subunit cRNAs at a ratio of 1:1 (Aa) show slower decay of elicited currents and a longer decay time constant for heteromeric receptors (Aa and b). The scale bars represent 1 sec and 1 μA for the α-nAChR response (black trace) and I see and 100 nA for the α7β2-nAChR response, thus also showing that current amplitudes were lower for heteromeric than for homomeric receptors. B: Normalized, mean (±SE), peak current responses (ordinate) of the indicated numbers of oocytes heterologously expressing nAChR α7 and β2 subunits (▪, ) or only α7 subunits (▴) as a function of time (abscissa, min) during challenges with choline alone (▪) or in the presence of 10 nM Aβ (, ▴) show sensitivity to functional block by Aβ only for heteromeric receptors. *p<0.05, **p<0.01, and ***p<0.001.

FIG. 8 depicts kinetics, pharmacology and Aβ sensitivity of α7-containing-nAChRs in nAChR β2 subunit knockout mice. Genotype analyses demonstrated that nAChR β2 subunits are not expressed in nAChR β2 knockout mice (A), whereas Lac-Z (as a marker for the knockout) was absent in wild-type (WT) mice (B). Kinetic analyses showed that whole-cell current kinetics and amplitudes differed for MS/DB neurons from WT compared to nAChR β2 subunit knockout homozygote mice (Ca,b). Compared to MS/DB neurons from WT mice (Da), choline-induced currents in MS/DB (Db) neurons from β2 knockouts were insensitive to DHβE but retained sensitivity to MLA (Dc). 1 nM Aβ₁₋₄₂ suppressed choline-induced currents in MS/DB neurons from WT (▪) but not from β2 knockout () mice (E). ‘Control’ responses (▴) were choline-induced currents in neurons from WT mice without exposure to Aβ₁₋₄₂. *p<0.05, **p<0.01.

FIG. 9 depicts atomic force microscopic (AFM) images of different forms of Aβ1-42. A: Images and B: height distribution analysis of Aβ1-42 at 0, 2 and 4 h following stock solution preparation showing time-dependent increase in Aβ aggregation. C: Aβ1-42 (diluted to 100 nM as stock solutions) was prepared using different protocols to obtain AFM imaging-confirmed, monomeric, oligomeric or fibriliar forms.

FIG. 10 depicts effects of 1 nM Aβ1-42 on ligand-gated ion channel activity in rat MS/DB neurons. A: typical whole-cell current response traces (left-to-right) before, after 6 or 10 min of exposure to 1 nM Aβ1-42, or after washout of peptide on 0.1 mM GABA-(a), 1 mM glutamate-(Glu, b), or 1 mM ACh-(c) induced currents. B. Mean (±SEM) normalized peak current responses (ordinate) as a function of time (abscissa, min; Aβ exposure from 0-10 min) from 4-12 neurons to 1 mM ACh (), 1 mM glutamate (Glu; ▴) or 0.1 mM GABA (▪). *p<0.05, **p<0.01.

FIG. 11 depicts pharmacological profiles for nAChR antagonist action at heterologously expressed α7- or α7β2-nAChRs in oocytes. Concentration-dependent block by MLA (at the indicated concentrations in nM after pre-exposure for 2 min indicated by open bars) of 10 mM choline-induced (applied as indicated by closed bars) whole-cell currents (representative traces shown) elicited in oocytes injected with nAChR α7 and β2 subunit cRNA (A) or only with α7 subunit cRNA (B) was not significantly different (p>0.05, n=5, C). However, choline-induced currents in oocytes expressing α7β2-nAChRs (D) were more sensitive (F) to block by DHβE (at the indicated concentrations in μM after pre-exposure for 2 min and continued exposure during agonist application indicated by open bars) than currents mediated by homomeric α 7-nAChRs (E).

FIG. 12 depicts Aβ induced hippocampal neuron degeneration. A: DAPI staining shows a loss of cultured hippocampal neurons after exposure to 100 nM Aβ₁₋₄₂. B: 100 nM (oligomers) Aβ-induced cytotoxicity measured by cell LDH levels. In these experiments, the primary hippocampal neuron cultures were used. *p<0.05, **p<0.01. C: Nissl staining shows hippocampal neuron (CA1 region) loss in 10-month-old 3×Tg-AD mice compared to aged-matched WT mice. There is ˜5% neuron loss at 3×Tg-AD hippocampus.

FIG. 13 depicts a significant impairment of hippocampal LTP in 3×TgAPP mice compared to WT mice. These mice were 12 months old when recording was performed. Schaffer collateral/CA1 LTP was induced by theta-burst stimulation.

FIG. 14 depicts Aβ up-regulation α7-nAChRs in hippocampal neurons. Quantitative RT-PCR showed that Aβ (10 or 100 nM) did not alter α7 subunit mRNA expression level in cultured hippocampal neurons (A) but notably increased α7 subunit mRNA expression in adult (10 month-old) 3×Tg mice (B) compared to WT mice. Altered levels of nAChR mRNA were normalized to untreated neurons (dashed line). Data in each group were internally normalized to GAPDH mRNA expression. C: [125 I]α-Bgt binding experiments showed that chronic exposure to Aβ increased α7-nAChR expression. D: Representative traces of choline-induced current responses in cultured Hippocampal neurons treated (right panel) and untreated (left panel) with Aβ. The numbers at the left side of traces represent the concentrations of choline. The holding potential was −60 mV. E: Bar graph compares 10 mM choline-induced currents in hippocampal neurons treated and untreated with Aβ.

FIG. 15 depicts hippocampal neuronal hyperexcitation induced by application of Aβ₁₋₄₂ oligomers for 10 days. A: Hyperpolarizing current induced sag-like membrane potential change (H-current) in cultured pyramidal neurons. B: Comparing neuronal spontaneous AP firing treated (Aa) and untreated (Ab) with Aβ. C: Comparing AP firing elicited by step current injections in the neurons treated and untreated with Aβ. D: Comparing input-output relationships between the neurons treated and untreated with Aβ. Each trace represents a typical case from 8-12 cells tested.

FIG. 16 depicts neural network hyperexcitation in 3×Tg-AD mice. A: Input-output curves in hippocampal CA1 slices from 3×Tg and WT mice. B: CCh (50 μM for 30 min)-induced θ-oscillations were observed in both 3×Tg-AD (Ba) and WT (Bb) mice, but 3×Tg-AD mice exhibited more synchronization, producing a higher frequency and more clustering bursts (C).

FIG. 17 depicts EEG recordings from 3×TgAPP and WT mice. The animals were free-moving and continuously monitored for one week. Two 3×TgAPP mice, but not WT mice, exhibited epileptic seizures.

FIG. 18 depicts roles of α7-nAChRs in chronic Aβ-induced neuronal hyper-excitation. A: α7-nAChR antagonist MLA (pretreated for 2 min) prevented Aβ-induced (oligomers for 9 days) expression of neural hyperexcitation. Traces Ab-d were recorded from the same neuron. B: Effects of Aβ on neuronal excitability in cultured hippocampal neurons prepared from α7−/− mice, and showed that genetic deletion of α7-nAChR prevented the induction of neuronal hyper-excitation. C: Roles of α7-nAChRs in Aβ-induced increase in sEPSCs. Chronic Aβ increased EPSC frequency (Cb, Da red column) but not amplitude (Db, red column), which was significantly prevented in α7−/− mice (Cd, Da black column). **, p<0.01. Each column was averaged from 6-8 cells tested.

FIG. 19 depicts CCh-induced network activity in WT and α7−/− slices. CCh (50 μM) was perfused throughout recording. In seven slices (from three WT mice), CCh induced both single field burst and θ-oscillations (A), while in four slices tested (from 3 α7−/− mice), CCh failed to induce θ-oscillations (B). Trace A and B were collected after perfusion of CCh for 30 min.

FIG. 20 depicts the roles played by α7-nAChRs in Aβ toxicity. α7−/− hippocampal neurons with Aβ₁₋₄₂ did not show toxic effects on these α7−/− hippocampal neurons compared to WT hippocampal neurons.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3_{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, the term “Aβ” refers to amyloid beta peptides.

As used herein, the term “nAChR” refers to nicotinic acetylcholine receptor.

As used herein, the term “Aβ₁₋₄₂” refers to amyloid beta peptides at positions 1-42 of the amyloid precursor protein (APP).

As used herein, the term “MS/DB” means medial septum/diagonal band.

As used herein, the term “AD” means Alzheimer's Disease.

As used herein, the term “dysfunctional signaling” refers to signaling mechanisms that are considered to be abnormal and not ordinarily found in a healthy subject or typically found in a population examined as a whole with an average amount of incidence.

As used herein, “treatment” or “treating” should be understood to include any indicia of success in the treatment, alleviation or amelioration of an injury, pathology or condition. This may include parameters such as abatement, remission, diminishing of symptoms, slowing in the rate of degeneration or decline, making the final point of degeneration less debilitating; improving a patient's physical or mental well-being; or, in some situations, preventing the onset of disease.

As used herein, “diagnose” or “diagnosis” refers to determining the nature or the identity of a condition or disease. A diagnosis may be accompanied by a determination as to the severity of the disease.

As used herein, “prognostic” or “prognosis” refers to predicting the outcome or prognosis of a disease.

As disclosed herein, nicotinic acetylcholine receptors (nAChRs) containing α7 subunits are believed to assemble as homomers. α7-nAChR function has been implicated in learning and memory, and alterations of α7-nAChR have been found in patients with Alzheimer's disease (AD). Findings in rodent, basal forebrain holinergic neurons are described herein consistent with a novel, naturally occurring nAChR subtype. In these cells, α7 subunits are coexpressed, colocalize, and coassemble with β2 subunit(s). Compared with homomeric α7-nAChRs from ventral tegmental area neurons, functional, heteromeric α7β2-nAChRs on cholinergic neurons freshly dissociated from medial septum/diagonal band (MS/DB) exhibit relatively slow kinetics of whole-cell current responses to nicotinic agonists and are more sensitive to the β2 subunit-containing nAChR-selective antagonist, dihydro-β-erythroidine (DH βE). Interestingly, heteromeric α7β2-nAChRs are highly sensitive to functional inhibition by pathologically relevant concentrations of oligomeric, but not monomeric or fibrillar, forms of amyloid β₁₋₄₂ (Aβ₁₋₄₂). Slow whole-cell current kinetics, sensitivity to DHβE, and specific antagonism by oligomeric Aβ₁₋₄₂ also are characteristics of heteromeric α7β2-nAChRs, but not of homomeric α7-nAChRs, heterologously expressed in Xenopus oocytes. Moreover, choline-induced currents have faster kinetics and less sensitivity to Aβ when elicited from MS/DB neurons derived from nAChR β2 subunit knock-out mice rather than from wild-type mice. The presence of novel, functional, heteromeric α7β2-nAChRs on basal forebrain cholinergic neurons and their high sensitivity to blockade by low concentrations of oligomeric Aβ₁₋₄₂ supports the existence of mechanisms for deficits in cholinergic signaling that could occur early in the etiopathogenesis of AD and could be targeted by disease therapies.

As described herein, the present invention provides a method of treating a neurodegenerative disorder in an individual, including providing a composition capable of inhibiting dysfunctional signaling of α7 nicotinic acetylcholine receptors (nAChRs), and administering a therapeutically effective amount of the composition to inhibit dysfunctional signaling of α7 nAChRs to treat the neurodegenerative disorder. In another method, the α7 nAChRs are heteromeric α7β2 nAChRs. In another embodiment, the composition is an α7β2 nAChR antagonist. In another embodiment, the composition is an β2 nAChR antagonist. In another embodiment, the composition is an α7 nAChR antagonist. In another embodiment, the composition is an an α7-nAChR positive allosteric modulator. In another embodiment, the composition is an antagonist of ionotropic glutamate receptors. In another embodiment, the neurodegenerative disorder is Alzheimer's Disease, dementia, Parkinson's Disease, and/or epilepsy. In another embodiment, the neurodegenerative disorder is an early stage form of Alzheimer's Disease. In another embodiment, the composition is a therapeutically effective amount of compound including kynurenic acid (KYNA), methyllycaconitine (MLA), α-bungarotoxin (BGT), cholinesterase inhibitor, memantine, and/or α-conotoxin, or a pharmaceutical equivalent, derivative, analog and/or salt thereof. In another embodiment, inhibiting the dysfunctional signaling of ac nAChRs includes restoring function of heteromeric α7β2 nAChRs. In another embodiment, inhibiting the dysfunctional signaling of α7 nAChRs includes protecting heteromeric α7β2 nAChRs from amyloid β (Aβ) effects. In another embodiment, inhibiting the dysfunctional signaling of α7-nAChRs includes a reduction in neuronal hyperexcitation. In another embodiment, inhibiting the dysfunctional signaling of α7 nAChRs includes a reduction in hyperexcitation of hippocampal neurons. In another embodiment, the individual is a human. In another embodiment, the individual is a rodent. In another embodiment, the dysfunctional signaling of α7 nAChRs occurs in the brain medial septum and/or diagonal band in the individual. In another embodiment, the dysfunctional signaling of α7 nAChRs occurs in the hippocampus in the individual.

As readily apparent to one of skill in the art, any number of readily available materials and known methods may be used to inhibit or activate nAChR signaling. For example, 7 nAChR antagonists such as α-conotoxin analogs (Armishaw, et al, Journal of Biological Chemistry, Vol. 285, No. 3; Armishaw, et al., Journal of Biological Chemistry, Vol. 284 No. 14), memantine (Aracava, et al., Journal of Pharmacology and Experimental Therapeutics, Vol. 312, No. 3), and kynurenic acid (Hilmas, et al., Journal of Neuroscience, 21(19): 7463-7473), may be used in conjunction with various embodiments herein to inhibit signaling of α7 containing nAChRs. Some examples include α7-nAChR antagonists, such as MLA α-bungarotoxin. Other examples include use of an α7-nAChR positive allosteric modulator, such as PNU-120596. Further examples include antagonists of ionotropic glutamate receptors, such as NBQX MK801.

In other embodiments, the present invention further provides a method of diagnosing a neurodegenerative disorder in an individual, including obtaining a sample from the individual, assaying the sample to determine the presence or absence of dysfunctional signaling of α7 nicotinic acetylcholine receptors (nAChRs) in the individual, and diagnosing the neurodegenerative disorder based on the presence of dysfunctional signaling of α7 nAChRs in the individual. In another embodiment, the α7 nAChRs are heteromeric α7β2 nAChRs. In another embodiment, the individual is a human. In another embodiment, the individual is a rodent. In another embodiment, the neurodegenerative disorder is Alzheimer's Disease, dementia, Parkinson's Disease, and/or epilepsy. In another embodiment, the dysfunctional signaling of α7 nAChRs occurs in the brain medial septum and/or diagonal band in the individual. In another embodiment, the dysfunctional signaling of α7 nAChRs occurs in the hippocampus in the individual. In another embodiment, the neurodegenerative disorder has proven non-responsive to treatment with galantamine, or a pharmaceutical equivalent, derivative, analog and/or salt thereof. In another embodiment, prior to obtaining the sample the individual is suspected of having a neurodegenerative disorder. In another embodiment, prior to obtaining the sample the individual demonstrates susceptibility to seizures. In another embodiment, prior to obtaining the sample the individual demonstrates abnormal θ oscillations.

in other embodiments, the present invention also provides a method of prognosing the onset of Alzheimer's Disease and/or dementia in an individual, including obtaining a sample from the individual, assaying the sample to determine the presence or absence of dysfunctional signaling of α7 nicotinic acetylcholine receptors (nAChRs) in the individual, and prognosing the onset of Alzheimer's Disease and/or dementia based on the presence of dysfunctional signaling of 7 nAChRs in the individual. In another embodiment, the α7 nAChRs includes heteromeric α7β2 nAChRs. In another embodiment, the dysfunctional signaling of α7 nAChRs occurs in the brain medial septum and/or diagonal band in the individual. In another embodiment, prior to obtaining the sample the individual is suspected of having a neurodegenerative disorder. In another embodiment, the dysfunctional signaling of α7 nAChRs occurs in the hippocampus in the individual. In another embodiment, prior to obtaining the sample the individual demonstrates susceptibility to seizures. In another embodiment, prior to obtaining the sample the individual demonstrates abnormal θ oscillations.

Other embodiments include a method of diagnosing an increased likelihood of developing a neurodegenerative disorder relative to a normal subject in an individual, including obtaining a sample from the individual, assaying the sample to determine the presence or absence of dysfunctional signaling of α7 nicotinic acetylcholine receptors (nAChRs) in the individual, and diagnosing an increased likelihood of developing the neurodegenerative disorder relative to a normal subject based on the presence of dysfunctional signaling of α7 nAChRs in the individual. In another embodiment, the α7 nAChRs are heteromeric α7β2 nAChRs. In another embodiment, the neurodegenerative disorder is Alzheimer's Disease, Parkinson's Disease, dementia and/or epilepsy, in another embodiment, prior to obtaining the sample the individual is suspected of having a neurodegenerative disorder. In another embodiment, prior to obtaining the sample the individual demonstrates susceptibility to seizures. In another embodiment, prior to obtaining the sample the individual demonstrates abnormal θ oscillations.

In one embodiment, the present invention provides a method of diagnosing susceptibility to a learning and/or memory disorder by determining the presence or absence of dysfunctional signaling of α7 containing nAChRs in a subject, where the presence of dysfunctional signaling of α7 containing nAChRs is indicative of susceptibility to the learning and/or memory disorder. In another embodiment, the α7 containing nAChRs are heteromeric α7β2-nAChRs. In another embodiment, the learning and/or memory disorder is Alzheimer's Disease. In another embodiment, the α7 containing nAChRs are found in basal forebrain cholinergic neurons. In another embodiment, the α7 containing nAChRs are found in the hippocampus. In another embodiment, the subject is a rodent. In another embodiment, the subject is a human.

In another embodiment, the present invention provides a method of diagnosing a learning and/or memory disorder by determining the presence or absence of dysfunctional signaling of α7 containing nAChRs in a subject, where the presence of dysfunctional signaling of α7 containing nAChRs is indicative of the learning and/or memory disorder. In another embodiment, the α7 containing nAChRs are heteromeric α7β2-nAChRs. In another embodiment, the learning and/or memory disorder is Alzheimer's Disease. In another embodiment, the α7 containing nAChRs are found in basal forebrain cholinergic neurons. In another embodiment, the α7 containing nAChRs are found in the hippocampus. In another embodiment, the subject is a rodent. In another embodiment, the subject is a human.

In one embodiment, the present invention provides a method of treating a learning and/or memory disorder in a subject by determining the presence of dysfunctional signaling of α7 containing nAChRs and inhibiting the dysfunctional signaling of α7 containing nAChRs. In another embodiment, the learning and/or memory disorder is Alzheimer's Disease. In another embodiment, inhibiting dysfunctional signaling of α7 containing nAChRs includes inhibiting expression of the nAChR α7 subunit. In another embodiment, inhibiting heteromeric α7β2-nAChR dysfunctional signaling includes the inhibition of expression of the nAChR β2 subunit. In another embodiment, the inhibition of expression of the nAChR β2 subunit includes fast whole-cell kinetics and/or low sensitivity to amyloid beta peptides.

In various embodiments, the present invention provides pharmaceutical compositions including a pharmaceutically acceptable excipient along with a therapeutically effective amount of compound that results in the inhibition of dysfunctional signaling of nAChRs. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration, “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Typical dosages of an effective composition that results in the inhibition of dysfunctional signaling of nAChRs can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

In other embodiments, the present invention also provides a kit to diagnose and/or treat a neurodegenerative disorder. The kit is an assemblage of materials or components, including at least one of the inventive compositions, such as a nucleotide or antibody detecting an α7 nicotinic acetylcholine receptor (nAChRs) associated transcript or protein, including subunits of α7 nAChRs, or signaling molecules related to nAChR function. In one embodiment, the α7 nAChRs are heteromeric α7β2 nAChRs. In another embodiment, the neurodegenerative disorder is Alzheimer's Disease, dementia, Parkinson's Disease and/or epilepsy. In another embodiment, the kit is disposable.

In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to apply progesterone topically. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generally

Nicotinic acetylcholine receptors (nAChRs) containing α7 subunits are believed to assemble as homomers. α7-nAChR function has been implicated in learning and memory, and alterations of α7-nAChR have been found in patients with Alzheimer's disease (AD). Findings in rodent, basal forebrain holinergic neurons are described herein consistent with a novel, naturally occurring nAChR subtype. In these cells, α7 subunits are coexpressed, colocalize, and coassemble with β2 subunit(s). Compared with homomeric α7-nAChRs from ventral tegmental area neurons, functional, heteromeric α7β2-nAChRs on cholinergic neurons freshly dissociated from medial septum/diagonal band (MS/DB) exhibit relatively slow kinetics of whole-cell current responses to nicotinic agonists and are more sensitive to the β2 subunit-containing nAChR-selective antagonist, dihydro-β-erythroidine (DH βE). Interestingly, heteromeric α7β2-nAChRs are highly sensitive to functional inhibition by pathologically relevant concentrations of oligomeric, but not monomeric or fibrillar, forms of amyloid β₁₋₄₂ (Aβ₁₋₄₂). Slow whole-cell current kinetics, sensitivity to DHβE, and specific antagonism by oligomeric Aβ₁₋₄₂ also are characteristics of heteromeric α7β2-nAChRs, but not of homomeric α7-nAChRs, heterologously expressed in Xenopus oocytes. Moreover, choline-induced currents have faster kinetics and less sensitivity to Aβ when elicited from MS/DB neurons derived from nAChR β2 subunit knock-out mice rather than from wild-type mice. The presence of novel, functional, heteromeric α7β2-nAChRs on basal forebrain cholinergic neurons and their high sensitivity to blockade by low concentrations of oligomeric Aβ₁₋₄₂ supports the existence of mechanisms for deficits in cholinergic signaling that could occur early in the etiopathogenesis of AD and could be targeted by disease therapies.

### Example 2

### Acutely-Dissociated Neurons from the CNS and Patch-Clamp Whole-Cell Current Recordings

Neuron dissociation and patch clamp recordings were performed as described in (Wu et al., 2002; Wu et al., 2004b). Briefly, each postnatal 2-4 week-old Wistar rat or mouse (wild-type C57/Bl6 or nAChR β2 knockout mice on a C57/Bl6 background kindly provided by Dr. Marina Picciotto, Yale University) was anesthetized using isoflurane, and the brain was rapidly removed. Several 400-μm coronal slices, which contained the medial septum/diagonal band (MS/DB) or the ventral tegmental area (VTA), were cut using a vibratome (Vibratome 1000 plus; Jed Pella Inc., Redding, Calif.) in cold (2-4° C.) artificial cerebrospinal fluid (ACSF) and continuously bubbled with carbogen (95% O₂-5% CO₂). The slices were then incubated in a pre-incubation chamber (Warner his., Holliston, Mass.) and allowed to recover for at least 1 h at room temperature (22±1° C.) in oxygenated ACSF. Thereafter, the slices were treated with pronase (1 mg/6 mL) at 31° C. for 30 min and subsequently treated with the same concentration of thermolysin for another 30 min. The MS/DB or VTA region was micropunched out from the slices using a well-polished needle. Each punched piece was then dissociated mechanically using several fire-polished micro-Pasteur pipettes in a 35-mm culture dish filled with well-oxygenated, standard external solution (in mM: 150 NaCl, 5 KCl, 1 MgCl₂, 2 CaCl₂, 10 glucose 10, and 10 HEPES; pH 7.4 (with Tris-base). The separated single cells usually adhered to the bottom of the dish within 30 min. Perforated-patch whole-cell recordings coupled with a U-tube or two-barrel drug application system were employed (Wu et al., 2002). Perforated-patch recordings closely maintain both intracellular divalent cation and cytosolic element composition (Horn and Marty, 1988). In particular, perforated-patch recording was used to maintain the intracellular ATP concentration at a physiological level. To prepare for perforated-patch whole-cell recording, glass microelectrodes (GC-1.5; Narishige, East Meadow, N.Y.) were fashioned on a two-stage vertical pipette puller (P-830; Narishige, East Meadow, N.Y.), and the resistance of the electrode was 3 to 5 MD when filled with the internal solution. A tight seal (>2 GΩ) was formed between the electrode tip and the cell surface, which was followed by a transition from on-cell to whole-cell recording mode due to the partitioning of amphotericin B into the membrane underlying the patch. After whole-cell formation, an access resistance lower than 60 MΩ was acceptable during perforated-patch recordings in current-clamp mode, and an access resistance lower than 30 MΩ was acceptable during voltage-clamp recordings. The series resistance was not compensated in the experiments using dissociated neurons. Under current-clamp configuration, membrane potentials were measured using a patch-clamp amplifier (200B; Axon Instruments, Foster City, Calif.). Data was filtered at 2 kHz, acquired at 11 kHz, and digitized on-line (Digidata 1322 series A/D board; Axon instruments, Foster City, Calif.). All experiments were performed at room temperature (22±1° C.). The drugs used in the present study were GABA, glutamate, ACh, choline, methyllycaconitine (MLA), dihydro-β-erythroidine (DHβE), muscarine (all purchased from Sigma-Aldrich, St. Louis, Mo.), RJR-2403 (purchased from Tocris Cookson Inc., Ballwin, Mo.), and Aβ₁₋₄₂ and scrambled Aβ₁₋₄₂ (purchased from rPeptide, Athens, Ga.).

### Example 3

### RT-PCR to Profile nAChR Subunit Expression in MS/DB

Riboprobe Construction:

Templates for in vitro transcription were created using PCR and sense or antisense primers spanning the 5′ SP6 promoter or the 3′ T7 promoter, respectively:

α7 subunit:
5′-atttaggtgacactatagaagnggatcatcgtgggcctctcagt
g-3′
5′-taatacgactcactatagggagagttggcgatgtagcggacct
c-3′
β2 subunit:
5′-atttaggtgacactatagaagngtcacggtgttcctgctgctcatc
t-3′
5′-taatacgactcactatagggagatcctccctcacactctggtcatc
a-3′.

Antisense or sense probes were then created by in vitro transcription using SP6 or T7 polymerases, respectively, and by incorporation of biotin-tagged UTP (for β2 subunit probes) or digoxigenin-tagged UTP (for α7 subunit probes; biotin or digoxigenin RNA labeling mix; Roche Applied Science, Indianapolis, Ind.). 433 bp or 520 bp products corresponded to mRNA nucleotides 953-1385 for α7 subunits or mRNA nucleotides 1006-1525 for β2 subunits thus produced are highly specific to the individual subunits.

Tissue RT-PCR:

RT-PCR assays followed by Southern hybridization with nested oligonucleotides were done as previously described to identify nAChR subunit transcripts and to quantify levels of expression normalized both to housekeeping gene expression and levels of expression in whole brain (Zhao et al., 2003; Wu et al., 2004b), but using primers designed to detect rat nAChR subunits. The Southern hybridization technique coupled with quantitation using electronic isotope counting (Instant Imager, Canaberra Instruments, Meridien, Conn.) yielded results equivalent to those obtained using real-time PCR analysis.

Single-Cell RT-PCR:

Precautions were taken to ensure a ribonuclease-free environment and to avoid PCR product contamination during patch-clamp recording and single-cell collection prior to execution of RT-PCR. Single-cell RT-PCR was performed using the Superscript III CellDirect RT-PCR system (invitrogen, Carlsbad, Calif.). Briefly, after whole-cell patch-clamp recording, single-cell content was harvested by suction into the pipette solution (˜3 μL) and immediately transferred to an autoclaved 0.2 mL PCR tube containing 10 μL of cell resuspension buffer and 1 μL of lysis enhancer. Single cells were lysed by heating at 75° C. for 10 min. Potential contaminating genomic DNA was removed by DNase I digestion at 25° C. for 6 min. After heat-inactivation of DNaseI at 70° C. for 6 min in the presence of EDTA, reverse transcription (RT) was performed by adding reaction mix with oligo(dT)20 and random hexamers and SuperScirptIII enzyme mix and then incubating at 25° C. for 10 min and 50° C. for 50 min. The reaction was terminated by heating the sample to 85° C. for 5 min. The PCR primers for glyceraldehyde-3-phosphate dehydrogenase (GAPDH) and nAChR α3, α4, α7, β2 and β4 subunits were designed using the Primer 3 internet server (MIT) and assuming an annealing temperature of ˜60° C. [nearest neighbor]. PCR was performed with 20 μL of hot-start Platinum PCR Supermix (Invitrogen, Carlsbad, Calif.), 3 μL of cDNA template from the RT step, and 1 μL of gene specific primer pairs (5 pmole each) with the following thermocycling parameters: 95° C. for 2 min; (95° C. for 30 s, 60° C. for 30 s, and 72° C. for 40 s) ×70 cycles, 72° C. for 1 min. PCR products were resolved on 1.5% TBE-agarose gels, and stained gels were used to visualize bands, employing digital photography and a gel documentation system to capture images.

### Example 4

### Tissue Protein Extraction, Immunoprecipitation, and Immunoblotting for Confirmation of nAChR α7 and β2 Subunit Co-Assembly

Tissues were Dounce homogenized (10 strokes) in ice-cold lysis buffer (1% (v/v) Triton X-100, 150 mM EDTA, 10% (v/v) glycerol, 50 mM Tris-HCl, pH 8.0) containing 1× general protease inhibitor cocktails (Sigma-Aldrich, St. Louis, Mo.). The lysates were transferred to microcentrifuge tubes and further solubilized for 30 min at 4° C. The detergent extracts (supernatants) were collected by centrifugation at 15,000 g for 15 min at 4° C., and protein concentration was determined for sample aliquots using bicinchoninic acid (BCA) protein assay reagents (Pierce Chemical Co., Rockford, Ill.). The detergent extracts were then precleared with 50 μL of mixed slurry of protein A-Sepharose and protein G-Sepharose (1:1) (Amersham Biosciences, Piscataway, N.J.) twice, each for 30 min at 4° C. For each immunoprecipitation, detergent extracts (1 mg) were mixed with 1 μg of rabbit anti-α7 antisera (H302) or rabbit IgG (as immunological control) (Santa Cruz Biotechnology, Inc., Santa Cruz, Calif.) and incubated at 4° C. overnight with continuous agitation. Protein A-Sepharose and protein G-Sepharose mixtures (50 μL) were added and incubated at 4° C. for 1 h. The beads were washed four times with ice-cold lysis buffer containing protease inhibitors. Laemmli sample buffer eluates were resolved by SDS-PAGE. Proteins were transferred onto Hybond ECL nitrocellular membranes (Amershan Biosciences, Sunnyvale, Calif.). The membranes were blocked with TBST buffer (20 mM Tris-HC (pH 7.6), 150 mM NaCl, and 0.1% (v/v) Tween 20) containing 2% (w/v) non-fat dry milk for at least 2 h and incubated with rat monoclonal anti-β2 antibody (mAb270; Santa Cruz, Calif.) or anti-α7 antisera (H302), respectively, at 4° C. overnight. After three washes in TBST, the membranes were incubated with goat anti-rat or goat anti-rabbit secondary antibodies (1:10,000) (Pierce Chemical Co., Rockford, Ill.) for 1 h and washed. The bound antibodies were detected with SuperSignal chemiluminescent substrate (Pierce Chemical Co., Rockford, Ill.).

### Example 5

### Expression of Homomeric and Heteromeric α7-Containing-nAChRs in Xenopus oocytes and Two-Electrode Voltage-Clamp Recording

cDNAs encoding rat α7 and β2 subunits were amplified by PCR with pfuUltra DNA polymerase and subcloned into an oocyte expression vector, pGEMHE, with T7 orientation and confirmed by automated sequencing. cRNAs were synthesized by standard in vitro transcription with T7 RNA polymerase, confirmed by electrophoresis for their integrity, and quantified based on optical absorbance measurements using an Eppendorf Biophotometer.

Oocyte Preparation and cRNA Injection:

Female Xenopus laevis (Xenopus I, Ann Arbor, Mich.) were anesthetized using 0.2% MS-222. The ovarian lobes were surgically removed from the frogs and placed in an incubation solution consisting of (in mM): 82.5 NaCl, 2.5 KCl, 1 MgCl₂, 1 CaCl₂, 1 Na₂HPO₄, 0.6 theophylline, 2.5 sodium pyruvate, 5 HEPES, 50 mg/mL gentamycin, 50 U/mL penicillin and 50 μg/mL streptomycin; pH 7.5. The frogs were then allowed to recover from surgery before being returned to the incubation tank. The lobes were cut into small pieces and digested with 0.08 Wunsch U/mL liberase blendzyme 3 (Roche Applied Science, Indianapolis, Ind.) with constant stirring at room temperature for 1.5-2 h. The dispersed oocytes were thoroughly rinsed with incubation solution. Stage VI oocytes were selected and incubated at 16° C. before injection. Micropipettes used for injection were pulled from borosilicate glass (Drummond Scientific, Broomall, Pa.). cRNAs encoding α7 or β2 at proper dilution were injected into oocytes separately or in different ratios using a Nanoject microinjection system (Drummond Scientific, Broomall, Pa.) at a total volume of ˜20-60 nL.

Two-Electrode Voltage-Clamp Recording:

One to three days after injection, an oocyte was placed in a small-volume chamber and continuously perfused with oocyte Ringer's solution (OR2), consisting of (in mM): 92.5 NaCl, 2.5 KCl, 1 CaCl₂, 1 MgCl₂ and 5 HEPES; pH 7.5. The chamber was grounded through an agarose bridge. The oocytes were voltage-clamped at −70 mV to measure ACh (or choline)-induced currents using GeneClamp 500B (Axon instruments, Foster City, Calif.).

### Example 6

### Immunocytochemical Staining

Dissociated MS/DB neurons were fixed with 4% paraformaldehyde for 5 min, rinsed three times with PBS, and treated with saponin (1 mg/mL) for 5 min as a permeabilizing agent. After rinsing four times with PBS, the neurons were incubated at room temperature in anti-choline acetyltransferase (ChAT) primary antibody (AB305; Chemicon International, Temecula, Calif.) diluted 1:400 in Hank's balanced salt solution (supplemented with 5% bovine serum albumin as a blocking agent) for 30 min. Following another three rinses with PBS, a secondary antibody (anti-mouse IgG; Sigma-Aldrich) was applied at room temperature for 30 min (diluted 1:100). After rinsing a final three times with PBS, the labeled cells were visualized using a Zeiss fluorescence microscope (Zeiss, Oberkochen, Germany), and images were processed using Photoshop (Adobe Systems Inc., San Jose, Calif.). For double immunolabeling of α7 and β2 subunits of nAChRs on single dissociated MS/DB neurons, the following antibodies were used: a rabbit antibody (AS-5631S, 1:400; R and D, Las Vegas, Nev.) against α7 subunit, a rat antibody against β2 subunit (Ab24698, 1:500; Abeam, Cambridge, Mass.), Alexa Fluor 594-conjugated anti-rabbit IgG, and Alexa Fluor 488-conjugated anti-rat IgG; (1:300; Molecular Probes, Calif.).

### Example 7

### Aβ Preparation and Determination/Monitoring of Peptide Forms

Aβ Preparation:

Amyloid β peptides (Aβ₁₋₄₂) were purchased from rPeptide Com (Athens, Ga.). As previously described (Wu et al., 2004a), some preparations involved reconstitution of Aβ peptides per vendor specifications in distilled water to a concentration of 100 μM, stored at −20° C., and used within 10 days of reconstitution. These thawed peptide stock solutions were used to create working dilutions (1-100 nM) in standard external solution before patch-clamp recording. Working dilutions were used within 4 hours before being discarded. Atomic force microscopy (AFM) was employed to define and analyze over time the morphology of prepared Aβ₁₋₄₂. Aliquots of freshly prepared samples of Aβ₁₋₄₂ diluted in standard external solution were spotted on freshly cleaved mica. After 2 min the mica was washed with 200 μL of deionized water, dried with compressed nitrogen, and completely air-dried under vacuum. Images were acquired in air using a multimode AFM nanoscope IIA system (Veeco/Digital Instruments, Plainview, N.Y.) operating in the tapping mode using silicon probes (Olympus, Center Valley, Pa.).

Protocols to Obtain Different Forms of Aβ₁₋₄₂:

Different conditions were utilized to specifically prepare monomeric, oligomeric or fibrillar forms of Aβ₁₋₄₂

Monomers:

Aβ₁₋₄₂ was reconstituted in DMSO to a concentration of 100 μM and stored at −80° C. For each use, an aliquot of stock sample was freshly thawed and diluted into standard extracellular solution as above just before patch recordings and used for no more than 4 h. This protocol yielded a predominant, monomeric form.

Oligomers:

Aβ₁₋₄₂ reconstituted in distilled water to a concentration of 100 μM and stored at −80° C. was used within 7 d of reconstitution. Aliquots diluted in standard extracellular solution and used within 4 hi yielded a predominantly oligomeric form.

Fibrils:

Aliquots of Aβ₁₋₄₂ stock solution (water dissolved to 100 μM) were thawed and incubated at 37° C. for 48 h at low pH (pH=6.0). Working stocks diluted in standard extracellular solution yielded a predominantly fibrillar form.

### Example 8

### Genotyping of the nAChR β2 Subunit Knockout Mice

Genomic DNA from mice newly born to heterozygotic, nAChR β32 subunit knockout parents was extracted from mouse tail tips using the QIAgen DNeasy Blood & Tissue Kit following the manufacture's protocol. PCR amplification of the nAChR β2 subunit or lac-Z (an indicator for the knockout) were performed using the purified genomic DNA as template and gene specific primer pairs (forward primer: CGG AGC ATT TGA ACT CTG AGC AGT GGG GTC GC; backward primer: CTC GCT GAC ACA AGG GCT GCG GAC; lac-Z forward primer: CAC TAC GTC TGA ACG TCG AAA ACC CG; backward primer: CGG GCA AAT AAT ATC GGT GGC CGT GG with annealing at 55° C. for 1 min and extension at 72° C. for 1 min for 30 cycles with GO Taq DNA polymerase (Promega, Madison, Wis.). PCR products were resolved on 1% agarose gels and stained for visualization before images were captured using digital photography.

### Example 9

### Identification of Cholinergic Neurons Dissociated from Basal Forebrain

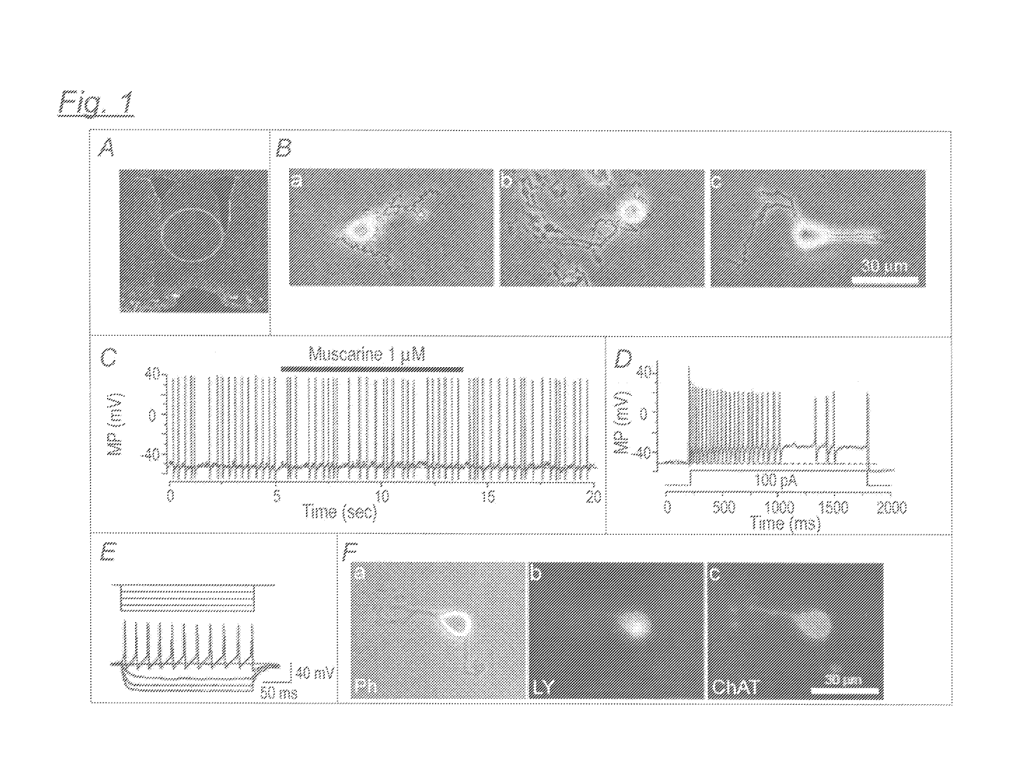

An initial series of experiments identified cholinergic neurons acutely dissociated from rat MS/DB (FIG. 1A). First, the cholinergic phenotype of acutely-dissociated neurons were identified from the MS/DB (FIG. 1Ba-c) based on published criteria (Henderson et al., 2005; Thinschmidt et al., 2005). In current-clamp mode, MS/DB neurons exhibited spontaneous action potential firing at low frequency (2.3±0.4 Hz, n=25 from 21 rats). This spontaneous activity was insensitive to the muscarinic acetylcholine receptor agonist, muscarine (1 μM) (FIG. 1C). Depolarizing pulses induced adaptation of action potential firing (FIG. 1D), and hyperpolarizing pulses failed to induce ‘sag’-like membrane potential changes (FIG. 1E). In some cases, the fluorescent dye lucifer yellow (0.5 mg/mL) was delivered into recorded cells after patch-clamp recordings, and choline acetyltransferase (ChAT) immunocytostaining was employed post-hoc (FIG. 1F). The presence of ChAT immunoactivity in recorded, dye-filled neurons confirmed that dissociated MS/DB neurons were cholinergic.

### Example 10

### Naturally-Occurring nAChRs in Rodent Forebrain Cholinergic Neurons

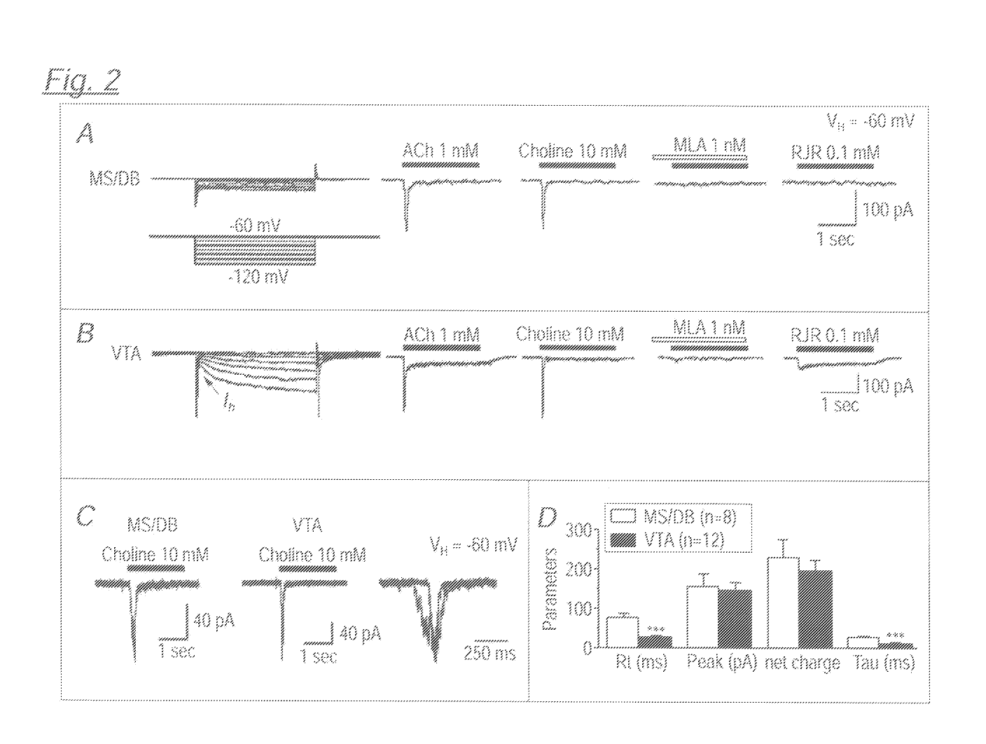

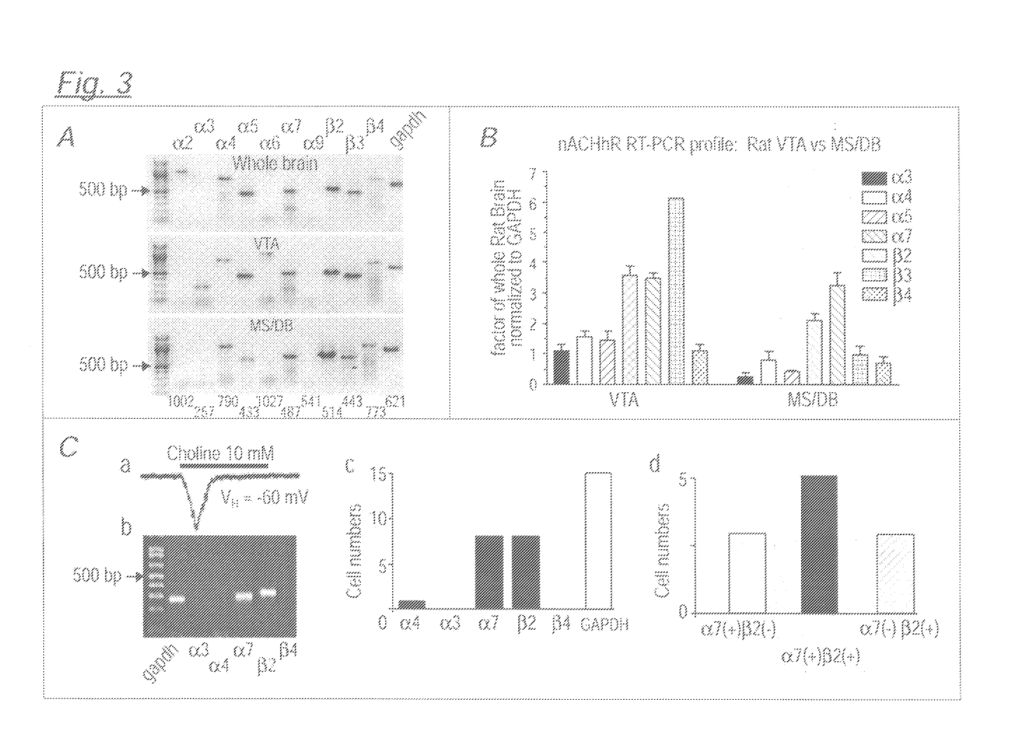

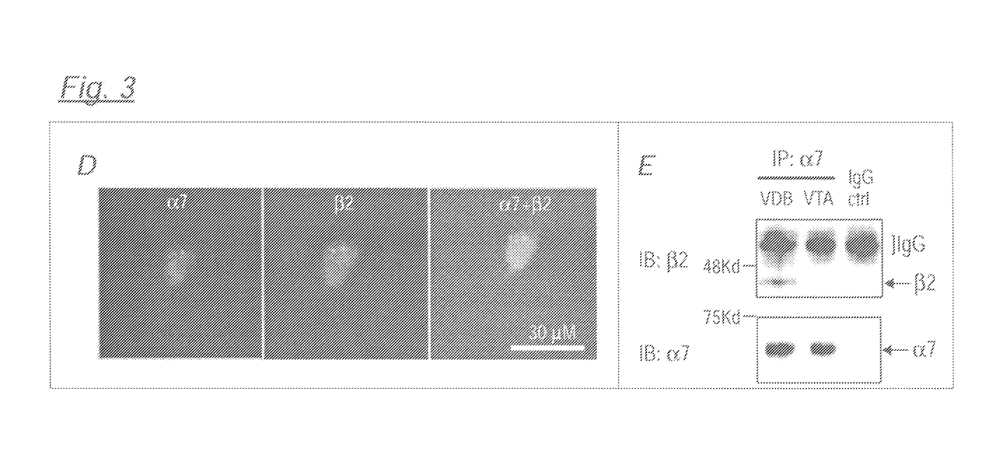

The inventors next tested for the presence of functional nAChRs on MS/DB cholinergic neurons. Under voltage-clamp recording conditions, rapid application of 1 mM ACh induced inward current responses with relatively rapid activation and desensitization kinetics (FIG. 2A). These ACh-induced responses were mimicked by application of the selective α7-nAChR agonist choline, blocked by the relatively-selective α7-nAChR antagonist methyllycaconitine (MLA), and insensitive to the relatively-selective α4β2-nAChR agonist RJR-2403 (FIG. 2A). Thus, the inward current evoked in MS/DB neurons had features similar to receptors containing α7 subunits. By contrast, in acutely-dissociated, dopaminergic (DAergic) neurons from the midbrain VTA, ACh-induced currents displayed a mixture of features that could be dissected pharmacologically and with regard to whole-cell current kinetics. Components of responses displaying slow kinetics and sustained, steady-state currents elicited by ACh were mimicked by RJR-2403, demonstrating that they were mediated by α4β2-nAChRs, whereas choline only induced transient peak current responses with very fast kinetics that are characteristic of homomeric α7-nAChRs (FIG. 2B). Interestingly, choline-induced currents in MS/DB cholinergic neurons exhibited relatively slow macroscopic kinetics than observed in VTA DAergic neurons (FIG. 2C). This impression was confirmed by quantitative analyses, which gave values for current rising time of 72.1±9.1 ms (n=8) for MS/DB neurons and 29.1±2.9 ms (n=12) for VTA neurons (p<0.001) and decay constants (tau, rate of decay from peak to steady state current) of 28.6±2.8 ms (n=8) for MS/DB neurons and 10.2±1.5 ms (n=12) for VTA neurons (p<0.001). There were no significant differences between either peak current amplitudes or net charge movements for responses elicited by choline in MS/DB or VTA neurons (FIG. 2D). These results demonstrated that functional nAChRs naturally expressed on rat MS/DB cholinergic neurons with some features like α7-nAChRs had slower whole-cell current kinetics than found for α7-nAChR-like responses in VTA DAergic neurons.

### Example 11

### Subunit Partnership for Naturally-Occurring nAChRs in Rodent Basal Forebrain Cholinergic Neurons

With regard to relatively slow kinetics of α7-nAChR-like responses in MS/DB cholinergic neurons due to co-assembly of α7 with other nAChR subunits, the inventors performed relative quantitative RT-PCR analysis of nAChR subunit expression as messenger RNA in MS/DB compared to whole-brain and VTA tissues. The results demonstrated that nAChR α7 and β2 subunits were among those co-expressed regionally (FIG. 3A, B). These studies were extended to single-cell RT-PCR analysis of nAChR subunit expression in acutely-dissociated neurons from the MS/DB used in patch-clamp recordings (FIG. 3Ca-c). Quantitative analysis indicated a high frequency of nAChR α7 and β2 subunit co-expression as message in recorded MS/DB neurons (FIG. 3Cd). Mindful of the current concerns about the specificity of all anti-nAChR subunit antibodies (Moser et al., 2007), nevertheless it was shown qualitatively, based on dual-labeling immunofluorescent staining (FIG. 3D), that α7 and β2 subunits were co-localized in many MS/DB neurons subjected to patch-clamp recording. More direct evidence for co-assembly of nAChR α7 and β2 subunit proteins came from co-immunoprecipitation studies using subunit-specific antibodies. Protein extracts from rat MS/DB or VTA tissues (collected from rats aged between 18-22 days) were subjected to immunoprecipitation (IP; FIG. 3E; left panel) with a rabbit anti-nAChR α7 subunit antibody (H302) or with rabbit IgG (as an immunological control) followed by immunoblotting (IB) with a rat anti-nAChR β2 subunit monoclonal antibody (mAb270). As indicated herein, the β2 subunit was readily detected immunologically in anti-α7 immunoprecipitates from MS/DB but not from VTA regions under our experimental conditions (FIG. 3E, upper left panel, lane 1 vs. 2). Reprobing the same blot with the rabbit anti-α7 antibody (H302) verified that similar amounts of α7 subunits were precipitated from both MS/DB and VTA regions (FIG. 3E, lower left panel, lanes 1 and 2). Thus, co-precipitation of nAChR α7 and β2 subunits appeared only in samples from the rat MS/DB but not from the VTA. Collectively, these results demonstrate that nAChR α7 and β2 subunits are most likely co-assembled, perhaps to form a functional nAChR subtype, in rodent basal forebrain cholinergic neurons.

### Example 12

### Pharmacological Profiles of Functional nAChRs in Rat Forebrain Cholinergic Neurons

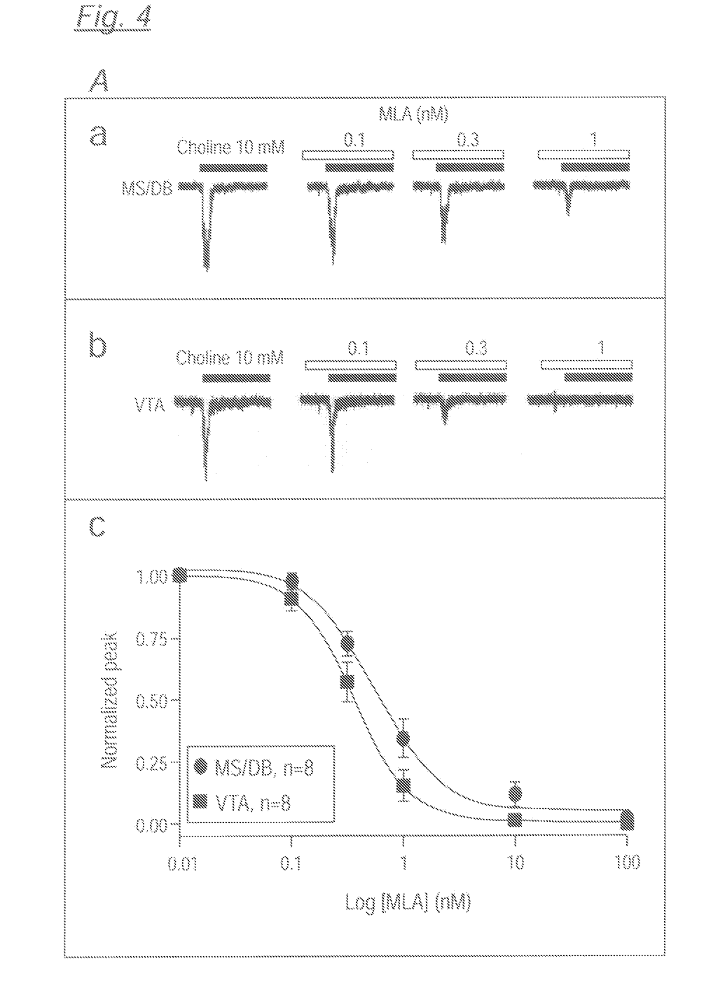

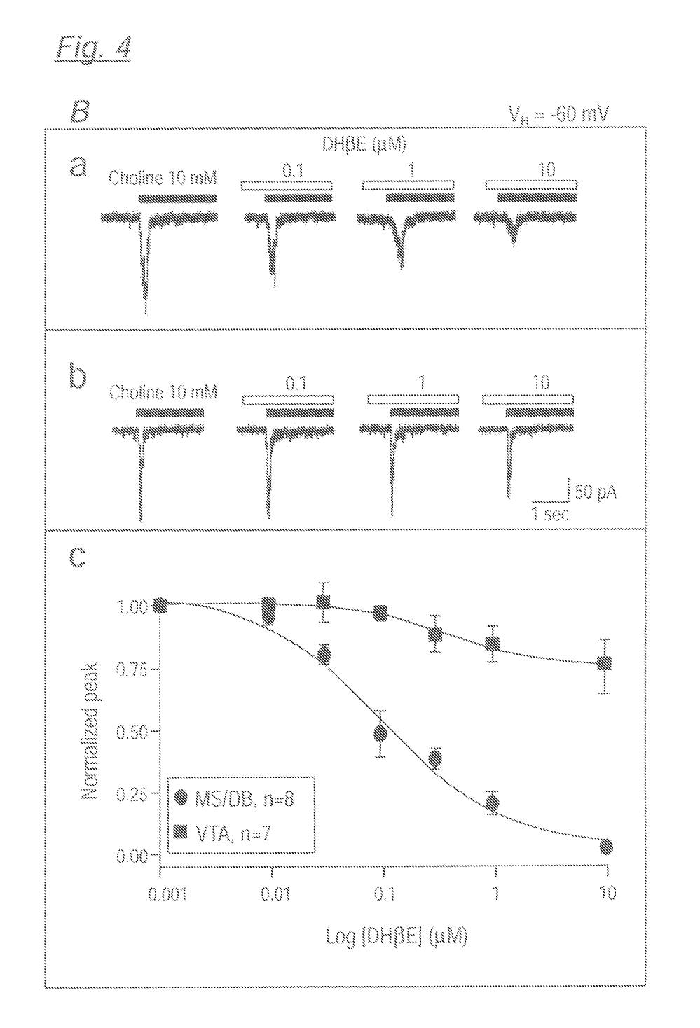

Pharmacological approaches were used to compare features of functional nAChRs in MS/DB cholinergic or VTA DAergic neurons. The α7-nAChR-selective antagonist, MLA showed similar antagonist potency toward choline-induced currents in either MS/DB (FIG. 4Aa) or VTA (FIG. 4Ab) neurons. Analysis of concentration-inhibition curves (FIG. 4Ac) yielded IC₅₀ values and Hill coefficients of 0.7 nM and 1.1, respectively, for MS/DB neurons (n=8) and 0.4 nM and 1.2, respectively, for VTA neurons (n=9, MS/DB vs. VTA p>0.05). However, the β2*-nAChR-selective antagonist, DHE was ˜500-fold less potent as an inhibitor of choline-induced current in MS/BD neurons (FIG. 4Ba) than in VTA neurons (FIG. 4Bb). IC₅₀ values and Hill coefficients for DHβE-induced inhibition were 0.17 μM and 0.9, respectively, for MS/DB neurons (n=8), and >100 μM and 0.3, respectively, for VTA neurons (n=7; MS/DB vs. VTA, p<0.001; FIG. 4Bc). These results are consistent with the concept that functional α7*-nAChRs on MS/DB cholinergic neurons also contain DH3E-sensitive β2 subunits.

### Example 13

### Functional nAChRs on Rat Basal Forebrain Cholinergic Neurons are Inhibited by Aβ₁₋₄₂

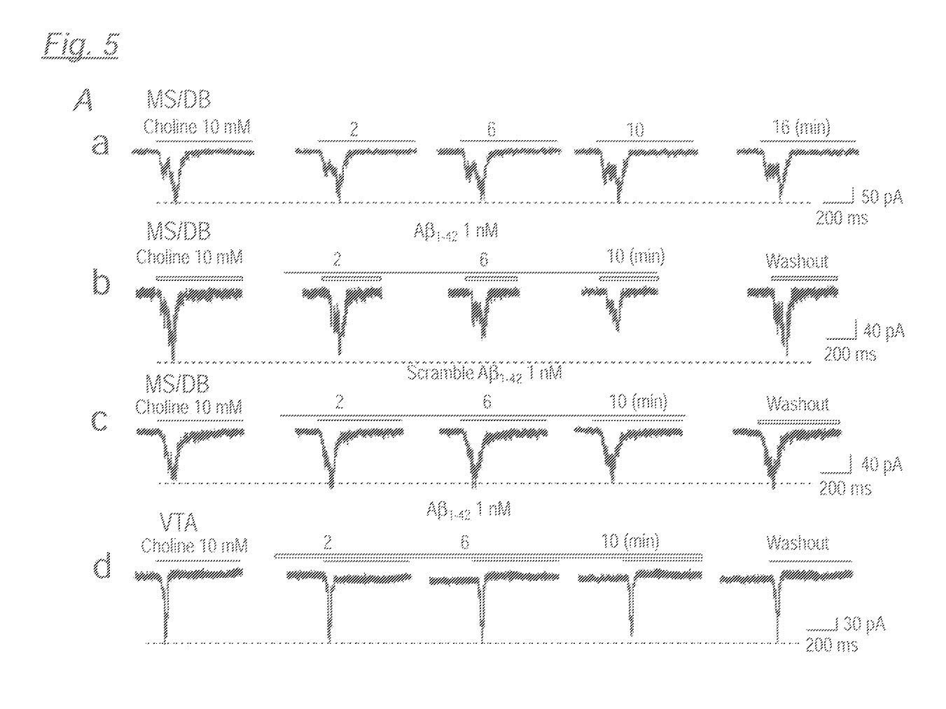

Basal forebrain cholinergic neurons are particularly sensitive to degeneration in AD. To demonstrate that novel α7β2-nAChRs on MS/DB cholinergic neurons are involved, the inventors determined the effects of Aβ₁₋₄₂ on these receptors. The experimental protocol involved repeated, acute challenges with 10 mM choline, and control studies in the absence of peptide demonstrated that there was no significant rundown of such responses when spaced at a minimum of 2-min intervals (FIG. 5Aa). During a continuous exposure to 1 nM Aβ₁₋₄₂ starting just after an initial choline challenge and continuing for 10 min, responses to choline challenges were progressively inhibited with time, although reversibly so as demonstrated by response recovery after 6 min of peptide washout (FIG. 5Ab). By contrast, exposure to 1 nM scrambled Aβ₁₋₄₂ (as a control peptide) had no effect (FIG. 5Ac). Choline-induced currents in dissociated VTA DAergic neurons were not sensitive to 1 nM Aβ₁₋₄₂ treatment (FIG. 5Ad). Quantitative analysis of several replicate experiments (FIG. 5B) confirmed that Aβ₁₋₄₂, even at 1 nM concentration, specifically inhibits putative α7β2-nAChR function on MS/DB cholinergic neurons but not function of homomeric α7-nAChRs on VTA DAergic neurons.

### Example 14

### Concentration- and Form-Dependent Inhibition by Aβ₁₋₄₂ of α7β2-nAChR Function on Basal Forebrain Cholinergic Neurons

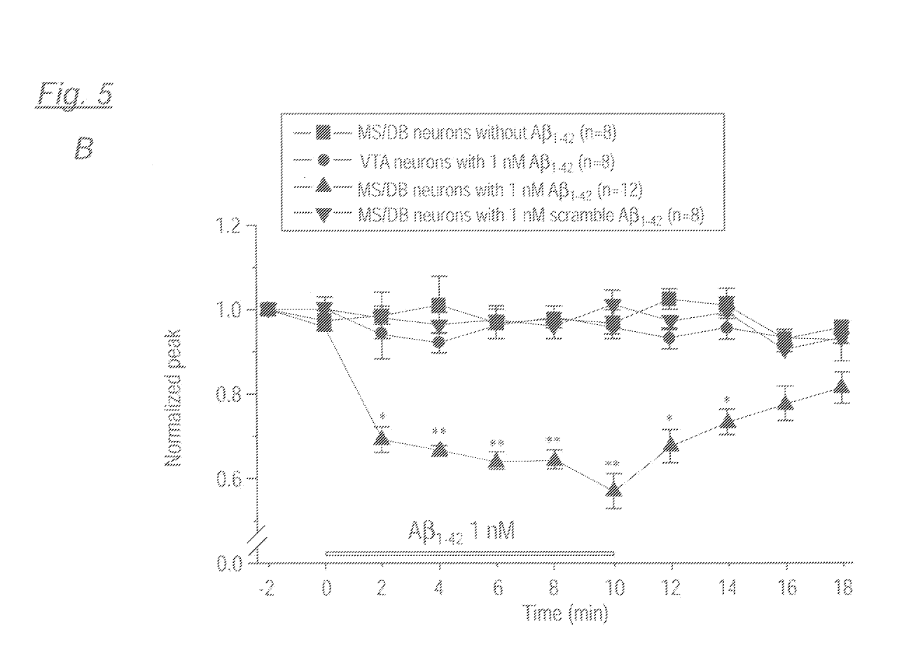

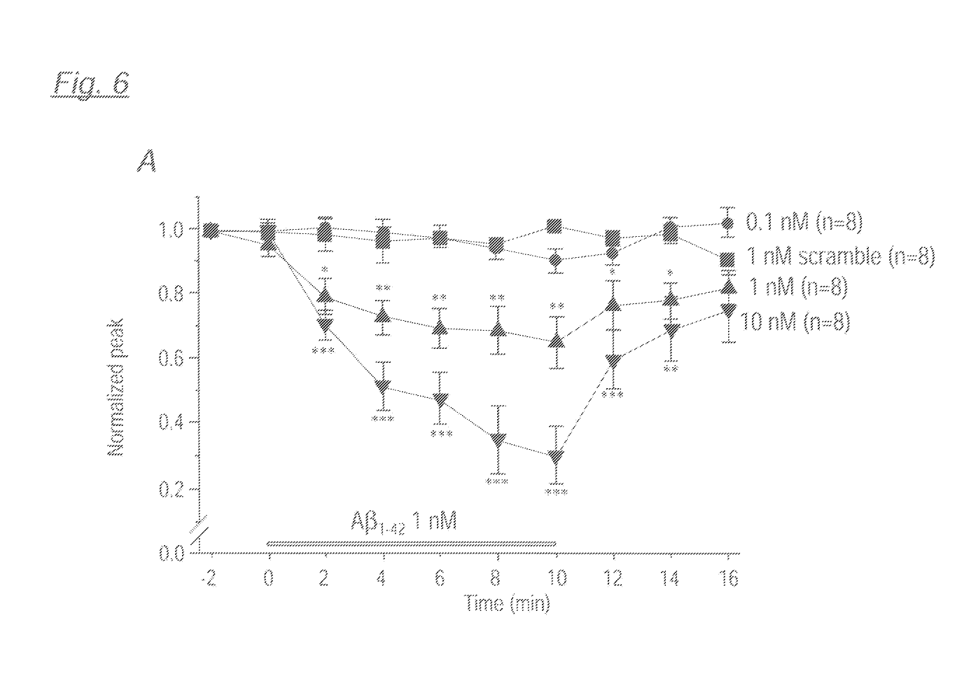

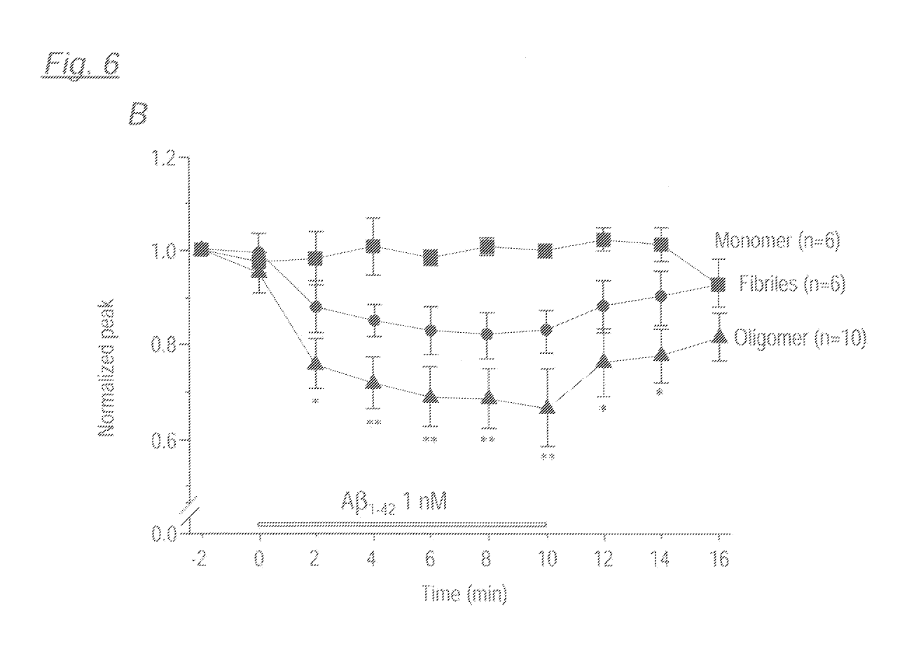

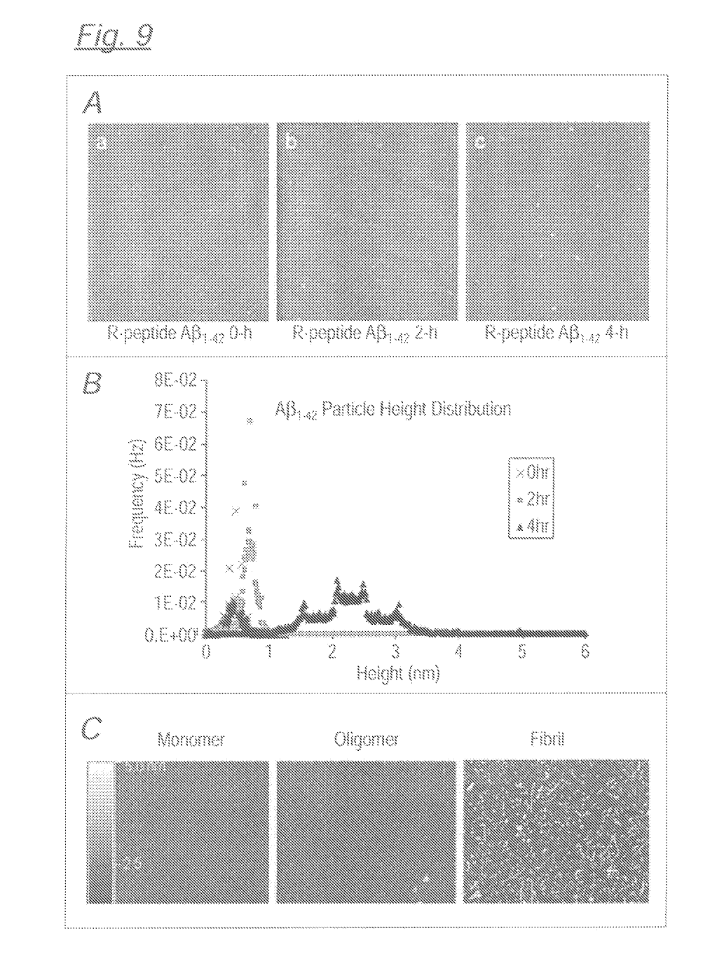

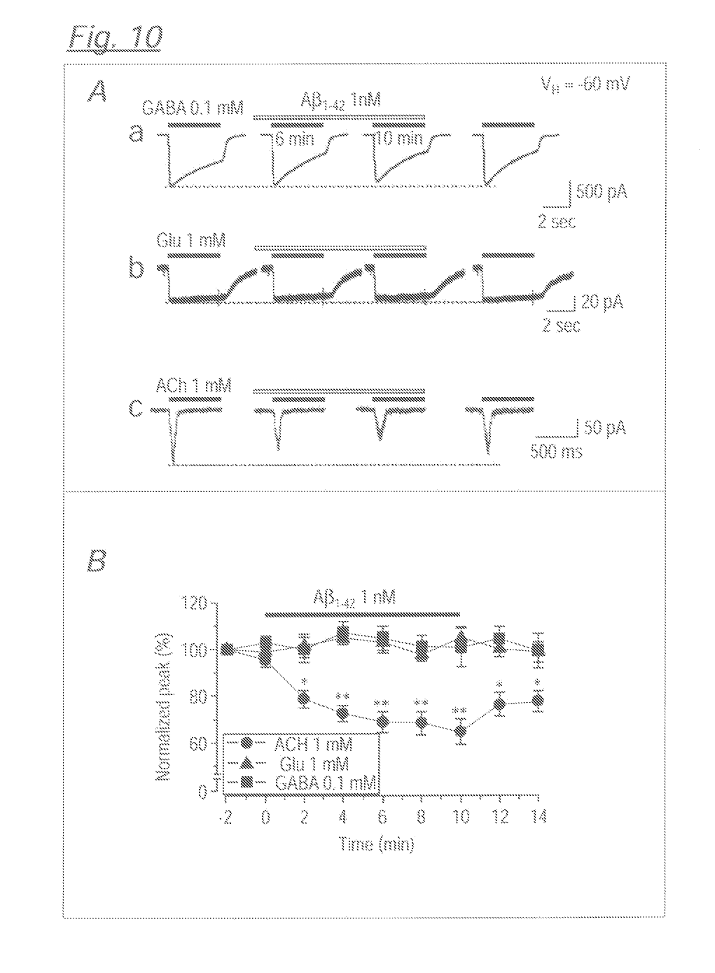

The inventors' previous studies indicated that α4β2-nAChRs were more sensitive to Aβ₁₋₄₂ than homomeric α7-nAChRs (Wu et al., 2004a). Concentration dependence of effects of Aβ₁₋₄₂ on choline-induced currents in MS/DB neurons was evident, with effects being negligible at 0.1 nM and effects at 1 nM being about half of those observed for 10 nM peptide (FIG. 6A). The magnitude of inhibition apparently had not yet reached maximum after 10 min of peptide exposure. The inventors also determined which form(s) of Aβ₁₋₄₂ showed the most potent inhibitory effect on choline-induced currents elicited in MS/DB neurons. Using different preparation protocols, the inventors produced Aβ₁₋₄₂ monomers (peptide dissolved in DMSO), oligomers (peptide dissolved in water), or fibrils (peptides dissolved in water at low pH (pH=6.0) and incubated at 37° C. for 2 days). Peptide forms were defined and monitored using AFM (see FIG. 9). At 1 nM, oligomeric Aβ₁₋₄₂ exhibited the greatest suppression of choline-induced responses, fibrillar Aβ had weaker inhibitory effect, and monomeric Aβ₁₋₄₂ failed to suppress choline-induced responses, indicating form-selective, Aβ₁₋₄₂ inhibition of nAChRs in MS/DB cholinergic neurons. To test whether Aβ₁₋₄₂ specifically inhibits nAChRs, the inventors also examined the effects of 1 nM Aβ₁₋₄₂ on GABA- or glutamate-induced currents in rat MS/DB cholinergic neurons, and the results demonstrated that both GABA_{A} receptors and ionotropic glutamate receptors were insensitive to inhibition by 1 nM Aβ₁₋₄₂ even when peptide effects on ACh-induced current were evident (FIG. 10). Collectively, these results indicate that, under our experimental conditions, pathologically-relevant, low nM concentrations of Aβ₁₋₄₂, especially in an oligomeric form, specifically inhibit function of apparently heteromeric α7β2-nAChRs, but peptides cannot inhibit function of homomeric α7-nAChRs, GABA_{A}, or glutamate receptors on MS/DB cholinergic neurons.

### Example 15

### Heteromeric α7β2-nAChRs Heterologously Expressed in Xenopus oocytes Display Slower Current Kinetics and High Sensitivity to Aβ₁₋₄₂

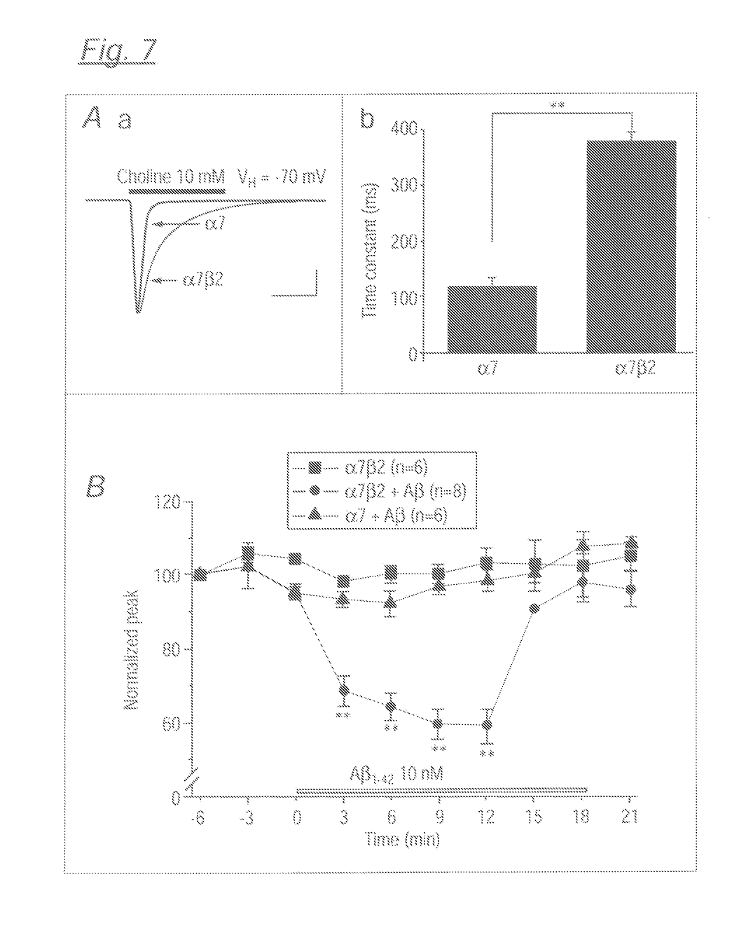

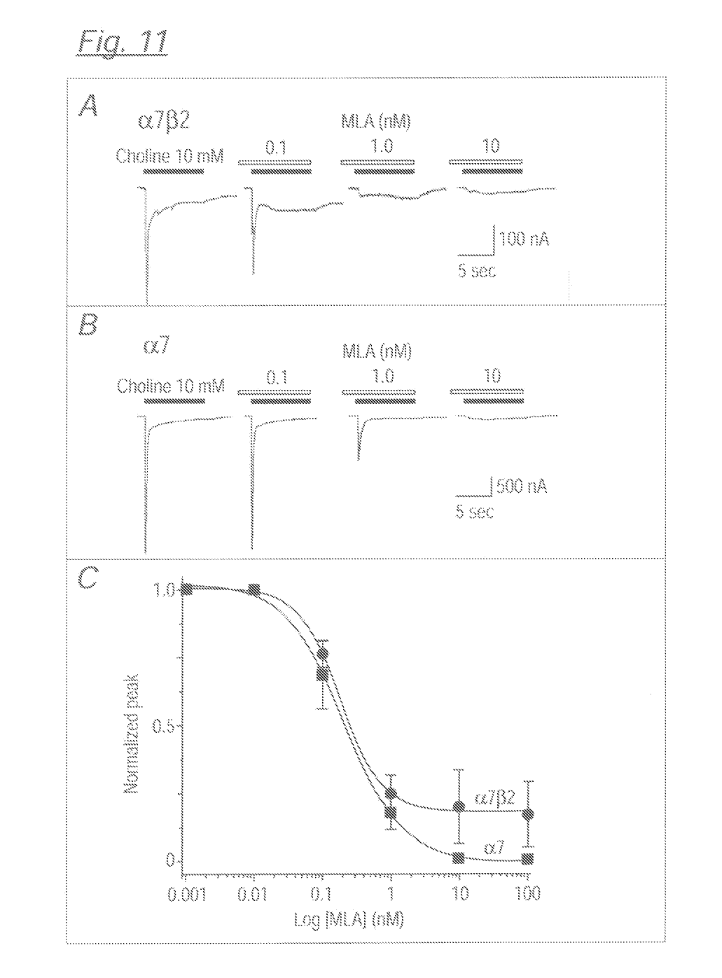

To further investigate features of presumed, novel α7β2-nAChRs as naturally expressed in basal forebrain cholinergic neurons, the inventors introduced nAChR α7 subunits alone or in combination with β2 subunits into Xenopus oocytes. Compared to homomeric α7-nAChRs (FIG. 7Aa), heteromeric α7β2-nAChRs expressed in oocytes injected with rat nAChR α7 and β2 subunit cRNAs at a ratio of 1:1 exhibited smaller peak current responses to choline and slower current decay rates (FIG. 7Ab). These results are consistent with findings in a previous report (Khiroug et al., 2002). As was the case for comparisons between native nAChR responses in rat MS/DB or VTA neurons (FIG. 4), sensitivity to functional blockade by MLA was similar for heterologously expressed α7β2- or α7-nAChR (FIG. 11A-C). Also similar to the case for native nAChR, heterologously expressed α7β2-nAChR were more sensitive to blockade by DHβE than were homomeric α7-nAChR. (Wang et al., 2000) indicates presence of β2 subunits with α7 subunits in rodent MS/DB neurons. The inventors then tested the sensitivity of heterologously-expressed α7β2-nAChRs in oocytes to Aβ. As was the case for presumed, native α7β2-nAChRs on MS/DB neurons, heterologously-expressed heteromeric α7β2-nAChRs, but not homomeric α7-nAChRs, demonstrated sensitivity to Aβ₁₋₄₂ (10 nM) and insensitivity to 10 nM scrambled Aβ₁₋₄₂ (FIG. 7B). These results obtained using heterologously-expressed nAChRs again are consistent with the hypothesis that nAChR α7 and β2 subunits likely co-assemble and form a unique α7β2-nAChR that enhances receptor sensitivity to pathologically-relevant, low nM concentrations of Aβ₁₋₄₂.

### Example 16

### Basal Forebrain nAChRs in nAChR β32 Subunit-Null Mice do not Show Co-Immunoprecipitation of nAChR α7 and β2 Subunits, Exhibit Fast Whole-Cell Current Kinetics, and Show Low Sensitivity to Aβ₁₋₄₂

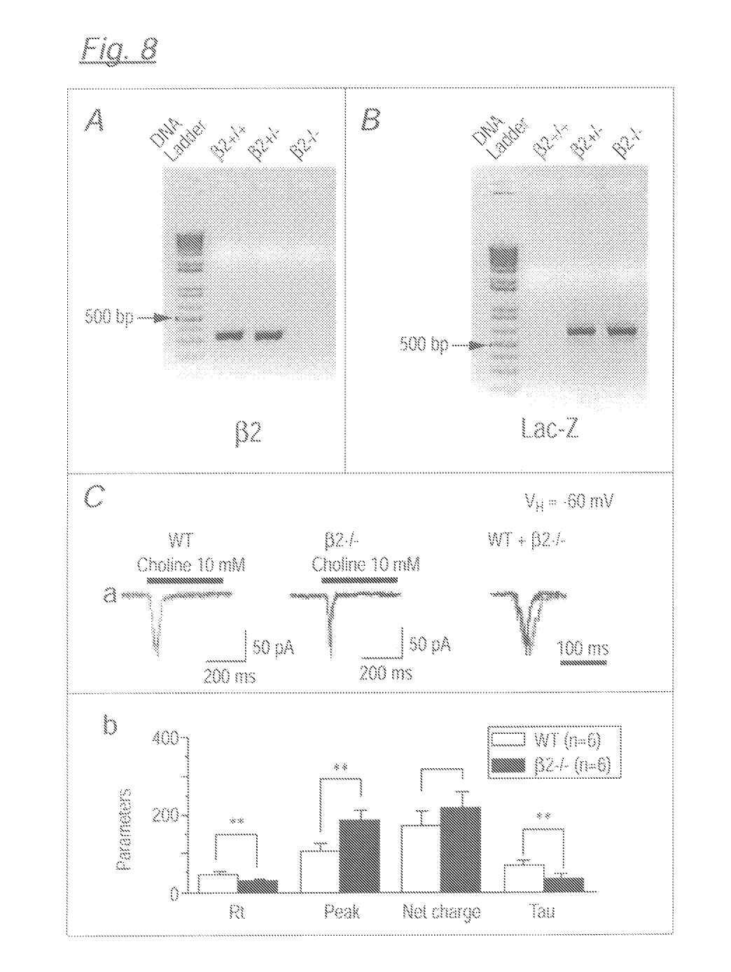

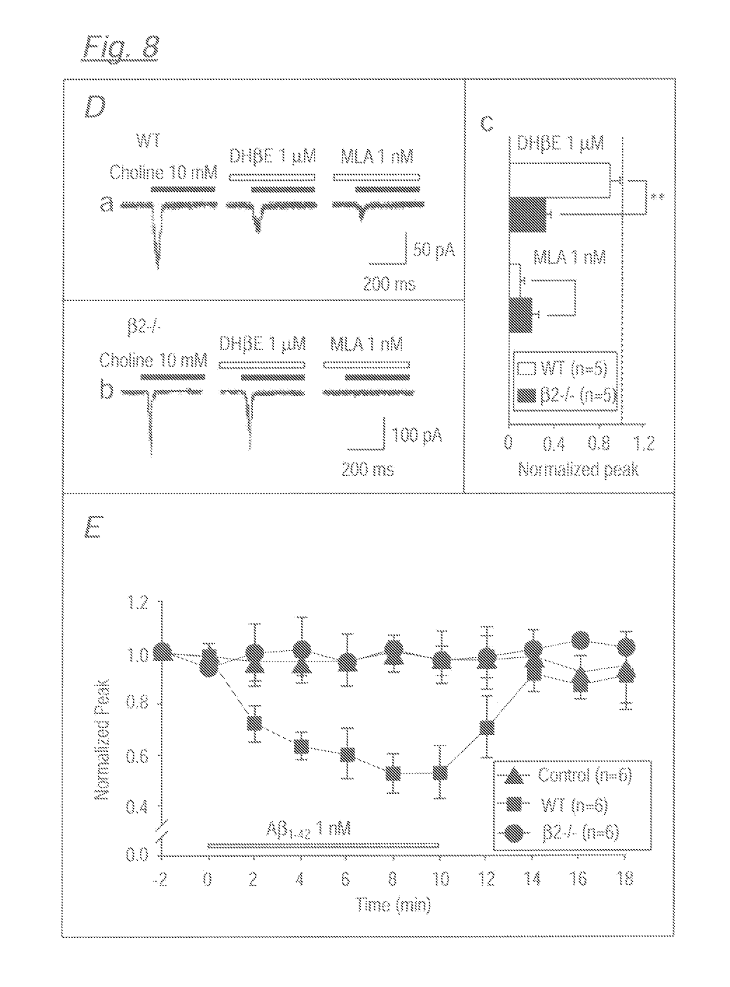

As further support for the concept that basal forebrain cholinergic neurons express novel α7β2-nAChRs, the inventors used wild-type and nAChR β2 subunit knockout (β2^{-/-}) mice. PCR genotyping was used to identify wild-type or β2^{-/-} mice (FIG. 8A, B). Using the immunoprecipitation protocol previously described and protein extracts from the MS/DB, nAChR β2 subunits were found to co-precipitate with nAChR α7 subunits only for samples from wild-type but not from β2^{-/-} mice (FIG. 3E, right panels). Choline-induced currents in MS/DB cholinergic neurons dissociated from β2^{-/-} mice exhibited higher current amplitude, faster kinetics (FIG. 8C), and lower sensitivity to DHβE (FIG. 8Da-c) than responses in cholinergic neurons dissociated from wild-type mice. As expected, 1 nM Aβ₁₋₄₂ failed to suppress choline-induced currents in MS/DB neurons from β2^{-/-} mice but did suppress choline-induced currents in MS/DB neurons from wild-type mice (FIG. 5E). These results again strongly support the concept that heteromeric, functional α7β2-nAChRs on basal forebrain MS/DB cholinergic neurons are highly sensitive to a pathologically-relevant concentrations of Aβ₁₋₄₂.

### Example 17

### Novel, Heteromeric, Functional α7β2-nAChR Subtype

nAChRs in basal forebrain participate in cholinergic transmission and cognitive processes associated with learning and memory (Levin and Rezvani, 2002; Mansvelder et al., 2006). During the early stages of AD, decreases in nAChR-like radioligand binding sites have been observed (Burghaus et al., 2000; Nordberg, 2001), suggesting that nAChR dysfunction could be involved in AD pathogenesis and cholinergic deficiencies (Nordberg, 2001). Evidence indicates that enhancement of α7-nAChR function protects neurons against Aβ toxicity through any or some combination of a number of different mechanisms, as outlined previously (Sadot et al., 1996; Lahiri et al., 2002; Nagele et al., 2002; Geerts, 2005; Liu et al., 2007a). On the other hand, pharmacological interventions or diminished nAChR expression produces learning and memory deficits (Levin and Rezvani, 2002).

Findings described herein are consistent with the natural expression of a novel, heteromeric, functional α7β2-nAChR subtype on forebrain cholinergic neurons that is particularly sensitive to functional inhibition by a pathologically-relevant concentration (1 nM) of Aβ₁₋₄₂. Some previous studies investigating the acute effects of Aβ₁₋₄₂ on nAChRs examined receptors on neurons from regions other than the basal forebrain or that were heterologously expressed (Liu et al., 2001; Pettit et al., 2001; Grassi et al., 2003; Wu et al., 2004a; Lamb et al., 2005; Pym et al., 2005) and/or used Aβ peptides at concentrations (between 100 nM and 10 μM) that greatly exceed Aβ concentrations found in AD brain (Kuo et al., 2000; Mehta et al., 2000). Other studies identified α7-nAChR-like, ACh-induced currents in MS/DB cholinergic neurons using slice-patch recordings (Henderson et al., 2005; Thinschmidt et al., 2005) and characterized functional, non-α7-nAChRs using acutely-dissociated forebrain neurons (Fu and Jhamandas, 2003). Studies described herein combined whole-cell current recordings from acutely-dissociated neurons and investigation of MS/DB cholinergic neuronal nAChRs to identify functional nAChRs that have some features of receptors containing α7 subunits, but also found high sensitivity of these nAChRs to low concentrations of Aβ₁₋₄₂. Studies described herein are consistent with other previous findings and also indicate that functional α7β2-nAChRs can be heterologously expressed in oocytes. Histological studies have demonstrated co-expression of nAChR α7 and β2 subunits in most forebrain cholinergic neurons (Azam et al., 2003). The results also are consistent with those observations and show cell-specific, co-expression of nAChR α7 and β2 subunits at both message and protein levels. There are other reports (Yu and Role, 1998); (Ei-Hajj et al., 2007) that nAChR α7 subunits could be co-assembled with other subunits to form native, heteromeric, α7*-nAChRs. These findings herein are consistent with those observations. The notion that the Aβ₁₋₄₂-sensitive, functional nAChR subtype in MS/DB neurons displaying some features of nAChRs containing α7 subunits, but distinctive from homomeric α7-nAChRs, is composed of α7 and β2 subunits, is supported by the loss of Aβ sensitivity and the conversion of functional nAChR properties to those like homomeric α7-nAChRs in nAChR β2 subunit knockout animals. It has been reported that there are two isoforms (α7-1 and α7-2) of α7-nAChR transcript in homomeric α7-nAChRs. The α7-2 transcript that contains a novel exon is widely expressed in the brain and showed very slow current kinetics (Severance et al., 2004); (Severance and Cuevas, 2004); (Saragoza et al., 2003). However, the inventors contend that the heteromeric α7β2-nAChR described in the present study and expressed in MS/DB neurons is not a homomeric nAChR composed of or containing the α7-2 transcript for three reasons: (1) in β2^{-/-} mice, α7-nAChR-like whole-cell current responses to choline acquire fast kinetic characteristics like those of α7-nAChR responses in VTA neurons, (2) immunoprecipitation-western blot analyses show co-assembly of α7 and β2 subunits from the MS/DB but not from the VTA, nor from the MS/DB of β2^{-/-} mice, and (3) pharmacologically heteromeric α7β2-nAChRs were sensitive not only to MLA, but also to DHβE.

A recent study suggested that levels of oligomeric forms of Aβ₁₋₄₂, rather than monomers or Aβ fibrils, most closely correlate with cognitive dysfunction in animal models of AD (Haass and Selkoe, 2007). The inventors' findings also convey that Aβ oligomers have the most profound effects on nAChR function, thus extending earlier studies of Aβ-nAChR interactions (Wu et al., 2004a) and illuminating why there have been apparent discrepancies in some of the earlier work concerning Aβ-nAChR interactions.

Alzheimer's disease (AD) is a dementing, neurodegenerative disorder characterized by accumulation of amyloid β (Aβ) peptide-containing neuritic plaques, degeneration of basal forebrain cholinergic neurons, and gradually impaired learning and memory (Selkoe, 1999). The extent of learning and memory deficits in AD is proportional to the degree of forebrain cholinergic neuronal degeneration, and the extent of Aβ deposition is used to characterize disease severity (Selkoe, 1999). Processes such as impairment of neurotrophic support and disorders in glucose metabolism have been implicated in cholinergic neuronal loss and AD (Dolezal and Kasparova, 2003). However, clear neurotoxic effects of Aβ across a range of in vivo and in vitro models suggest that Aβ plays potentially causal roles in cholinergic neuronal degeneration and consequent learning and memory deficits (Selkoe, 1999).

Based on the findings described herein, selective, high-affinity effects of oligomeric Aβ₁₋₄₂ on basal forebrain, cholinergic neuronal α7β2-nAChRs acutely contribute to disruption of cholinergic signaling and diminished learning and memory abilities (Yan and Feng, 2004). Moreover, to the extent that basal forebrain cholinergic neuronal health requires activity of α7β2-nAChRs, inhibition of α7β2-nAChR function by oligomeric Aβ₁₋₄₂ can lead to losses of trophic support for those neurons and/or their targets, and cross-catalyzed spirals of receptor functional loss and neuronal degeneration also can contribute to the progression of AD. Drugs targeting α7β2-nAChRs to protect them against Aβ effects or restoration of α7β2-nAChR function in cholinergic forebrain neurons will serve as viable therapies for AD.

### Example 18

### Aβ Accumulation and α7-nAChR Functional Dysregulation in AD Pathogenesis

The mechanisms of α7-nAChR-mediated toxic effects in AD mice are largely unknown and may be the result of Aβ upregulation of α7-nAChR expression and function, causing neural hyperexcitation and consequently, neurodegeneration. The traditional “Aβ concept” is that Aβ induces neurotoxicity and cholinergic neuronal degeneration, in turn causing synaptic impairment, and learning and memory deficits (Smith, et al., 2006; Viola et al., 2008, Nimmrich and Ebert, 2009). The clear, neurotoxic effects of Aβ across a range of in vivo or in vitro models suggests that Aβ plays a significant role in cholinergic neuronal degeneration and consequent learning and memory deficit. Other processes such as impairment of neurotrophic support and disorders of glucose metabolism have been implicated in cholinergic neuronal loss and AD (Dolezal and Kasparova, 2003) However, Aβ toxicity remains a significant factor underlying AD pathogenesis, based on Aβ accumulation and aggregation in neuritic or senile plaques and to the extent of Aβ deposition is a leading indicator for AD disease severity (Selkoe, 1999; Walsh and Selkoe 2004). Further elucidating the role of Aβ may improve AD diagnosis and treatment and focuses on the selective cholinergic neuronal deficits that are characteristic hallmarks of AD1 and the extent of learning and memory deficits in AD as proportional to the degree of forebrain cholinergic neuronal degeneration.

Based on the findings described herein, α7-nAChRs play an important role in the mediation of Aβ toxicity. More specifically, high α7-nAChR expression and/or function is present in AD. Further reports that activation of α7-nAChRs enhances cognitive function provides opportunity to consider application of α7-nAChR agonists to treat AD. However, emerging evidence has show that while Aβ inhibits α7-nAChRs acutely in most cases, these receptors actually exhibit enhanced expression, at the mRNA and protein level, in both AD patients and AD model animals (Jones et al., 2006; Counts et al., 2007b; Hellstron-Lindhal 2004a; Hellstron-Lindhal 2004b; Hellstron-Lindhal 1999; Dinley et al., 2002a; Dinley et al., 2002b; Chu et al., 2005; Teaktong et al., 2004). This may be due to the up-regulation of the receptor, based on an initial inhibitory state of α7-nAChRs by Aβ (Walsh and Selkoe, 2004). Long-term exposure to Aβ reverses this effect as shown by upregulation of 7-nAChRs in glial cells (Xiu et al., 2005; Yu et al., 2005). Other evidence demonstrates high-levels of Aβ causing neuronal or neurocircuit hyperexcitation. For example, chronic exposure to high levels of Aβ sensitizes some neuronal networks to hyperexcitation (Del Vecchio, et al., 2004). Over-expression of Aβ in animals models cause epileptiform activity within the entorhinal-hippocampal circuitry (Palop et al., 2007). Westmark et al., compared seizure threshold (test response to pentylenetetrazol, PTZ) between AD model animals (Tg2576) and wild-type mice, and found a reduction of seizure threshold in AD model animals, suggesting that Aβ induces neuronal hyperexcitation (Westermark et al., 2008). Together, without being bound by a particular theory, this suggests that the chronic effect of Aβ exposure could be α7-nAChRs hyper-expression, not hypo-expression and function. Determining these effects of chronic Aβ exposure on α7-nAChR function in hippocampal neurons in AD model animals or even in AD patients is essential to understanding the impact of α7-nAChRs in AD pathogenesis and therapy. Because AD patients (or model animals) exhibit hyper-expression and/or hyper-function of α7-nAChRs, using α7-nAChR antagonists to treat AD could have an important clinical impact (Counts et al., 2007; Dziewczapolski et al., 2009; Leonard and McNamara et al., 2007). The inventors contend that this provides an experimental basis for a new therapeutic strategy where appropriate attenuation, rather than potentiation, of α7-nAChRs may protect from, or even prevent Aβ toxicity in AD and consequently slow and/or improve learning and memory deficits.

### Example 19

### Effects Aβ on Hippocampal Neuron Degeneration

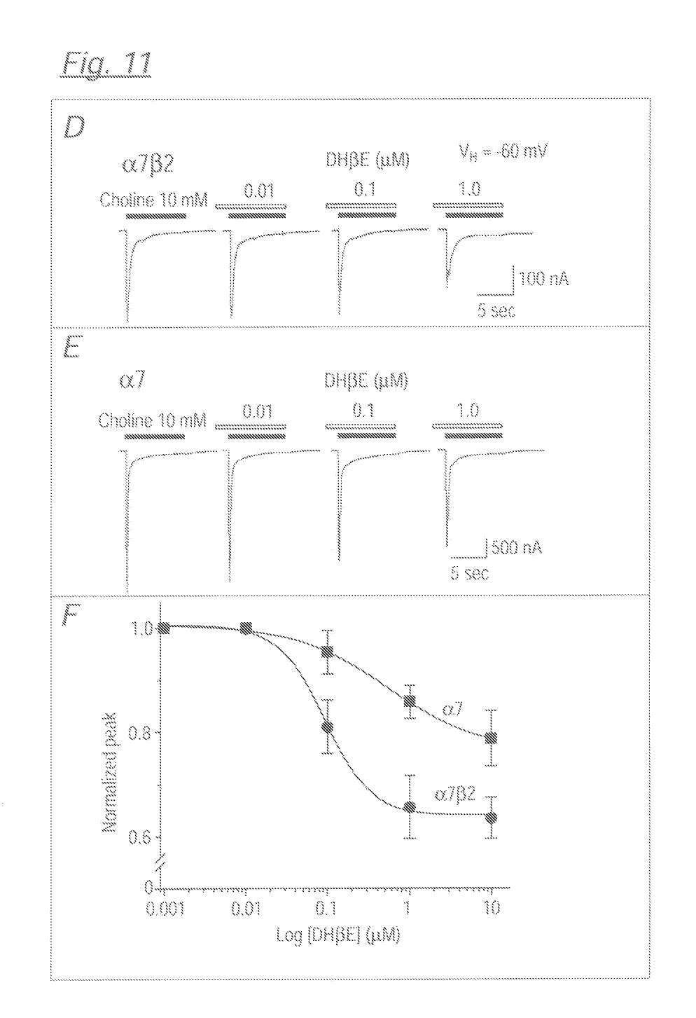

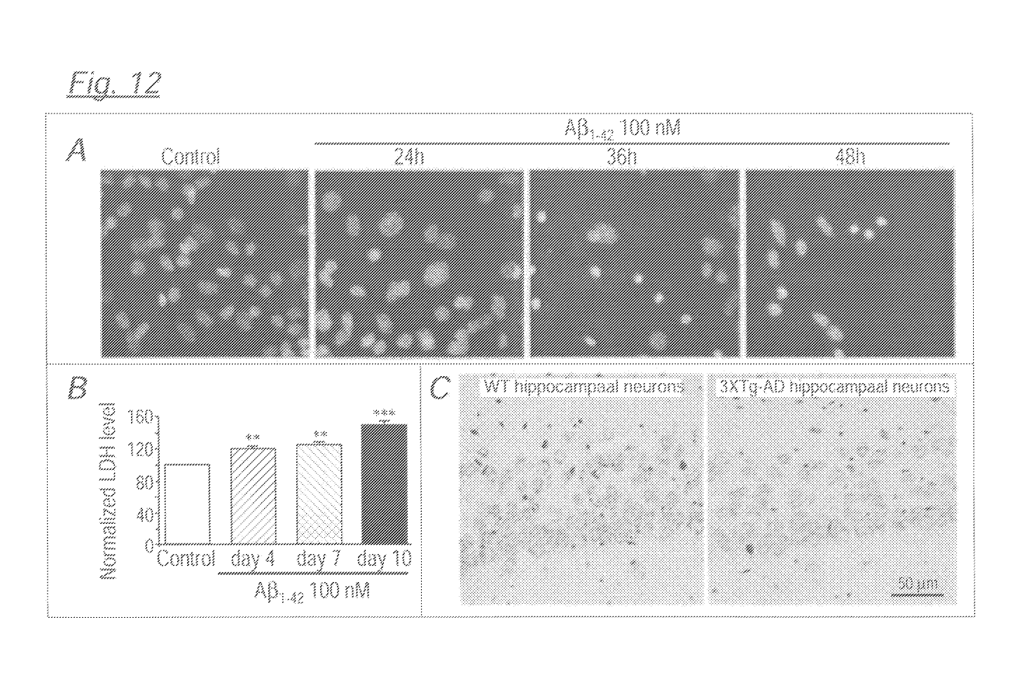

Experiments tested loss of cultured mouse hippocampal neurons treated with 100 nM Aβ₁₋₄₂ over several days, and the results showed that Aβ₁₋₄₂ treated neurons exhibited cell loss in an exposure-time-dependent manner, indicated by DAPI staining (FIG. 12A). Measurement of Aβ-induced cytotoxicity through assay of LDH levels show that in primary cultured hippocampal neurons, exposure to 100 nM Aβ 1-42 for 4, 7 and 10 days increased LDH levels, again in a manner dependent on exposure length (FIG. 12B). All these results suggested chronic Aβ exposure inducing hippocampal neuron degeneration. Testing hippocampal neuron loss in 3×Tg AD mice using Nissl staining showed neuron loss in AD mice compared to WT mice (FIG. 12C).

### Example 20

### Effects of Aβ on Hippocampal Synaptic Plasticity

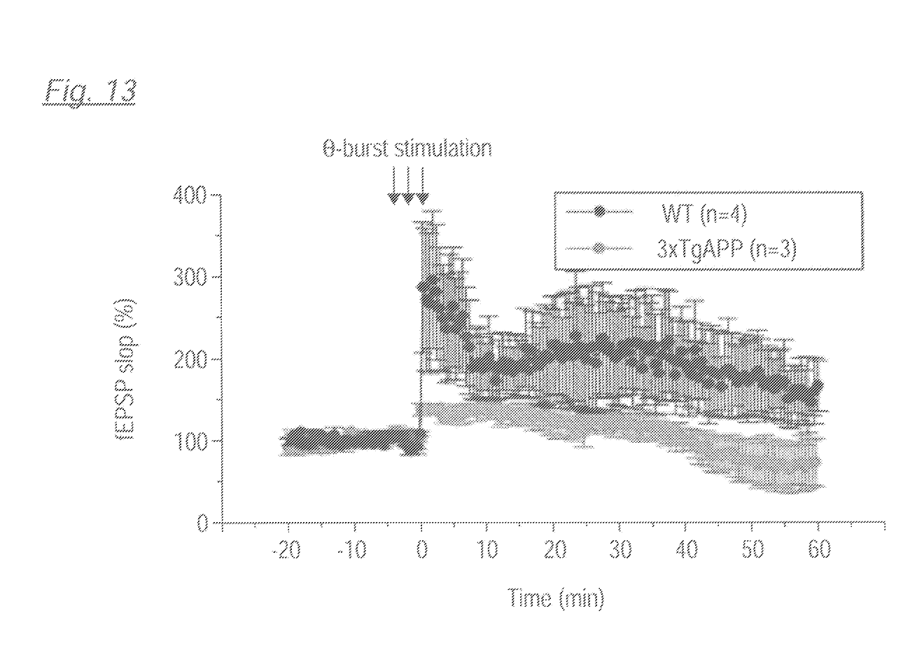

FIG. 13 shows a significant impairment of hippocampal long-term potentiation (LTP) in 3×TgAPP mice compared to WT mice. These mice were 12 months old when recording was performed. Schaffer collateral/CA1 LTP was induced by theta-burst stimulation.

### Example 21

### Aβ Up-Regulates α7-nAChRs in Hippocampal Neurons

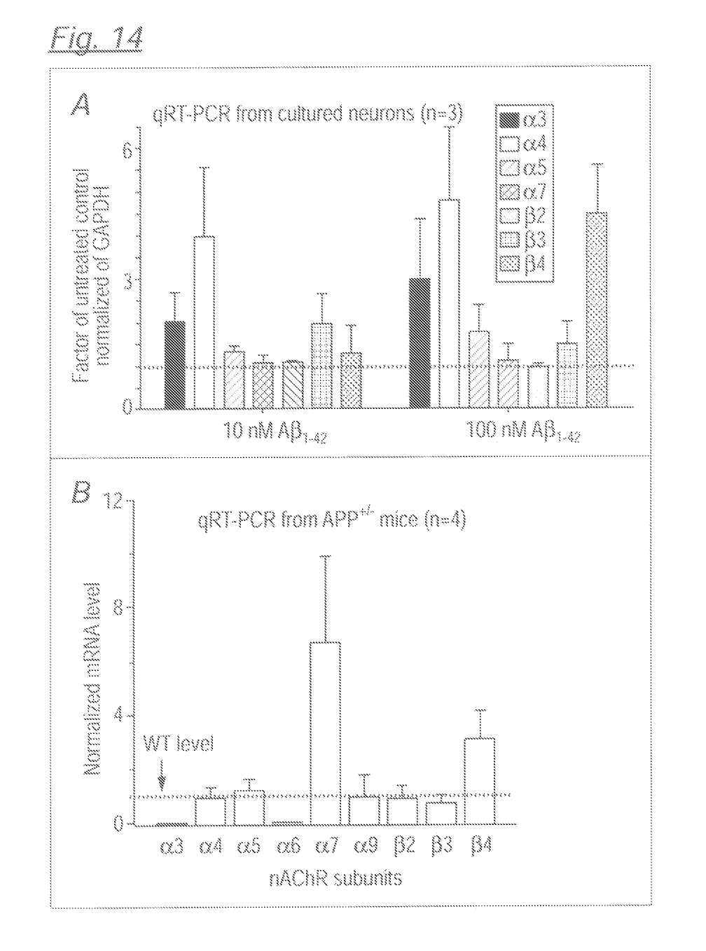

Further experiments demonstrate that that Aβ upregulates α7-nAChR expression and function. Quantitative RT-PCR experiments did not show significant difference of α7 mRNA expression between cultured neurons treated and untreated with 10 or 100 nM Aβ oligomers for 10 days (FIG. 14A), but in animals models showed a significant increase of α7 mRNA expression in aged (10 month-old) 3×TgAD mice compared to WT mice (FIG. 14B). Measurement of [125I]α-Bgt binding in cultured hippocampal neurons treated and untreated with 100 nM Aβ for 10 days, showed a significant increase of α7-nAChR binding after chronic Aβ exposure (FIG. 14C). Use of patch-clamp recording to measure 7-nAChR function in cultured hippocampal neurons treated and untreated with Aβ (100 nM for 10 days) showed that chronic treatment with 100 nM Aβ₁₋₄₂ substantially enhanced choline-induced currents compared to un-treated neurons (FIG. 14D,E). These results show that chronic exposure (9-10 days) to Aβ₁₋₄₂ up-regulates α7-nAChR expression and function, apparently through a posttranslational mechanism.

### Example 22

### Chronic Exposure to Aβ Induced Neuronal Hyperexcitation in Cultured Hippocampal Neurons

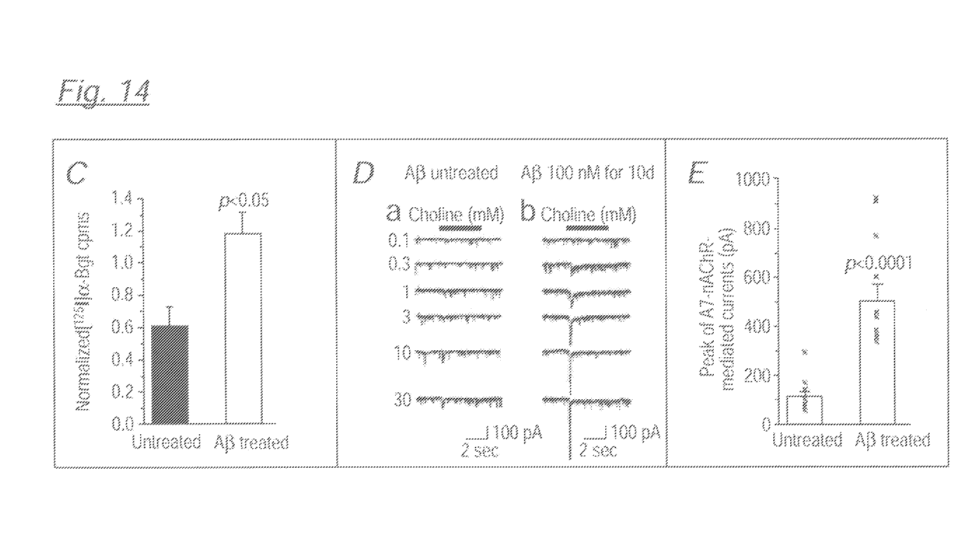

Recent reports demonstrate that hAPP mice exhibit hippocampal circuit hyperexcitation and epileptic seizures, but there is no direct evidence that Aβ induces these neuronal hyperexcitations (Amatniek et al., 2006; Palop et al., 2007; Alondon and Albuquerque, 1995). Using patch clamp methods, cultured hippocampal pyramidal neurons usually exhibit pyramidal shape, rare spontaneous action potential (AP) firing (FIG. 15Bb, black arrow), and had H-currents when holding potentials were altered from −60 to −120 mV (FIG. 15A, red arrow) (Alondon and Albuquerque, 1995). Compared to control (change culture medium daily, without Aβ₁₋₄₂ for 10 days, FIG. 15Bb), fibril form Aβ₁₋₄₂ treated neurons exhibited a more positive mean resting membrane potential (FIG. 15Ba, −53.4±2.6 mV, n=11 for treated neurons compared to −62.8±3.3 mV for untreated neurons, n=13, p<0.01). In addition, spontaneous bursting AP firing was observed in treated (FIG. 15Ba, red arrows) but not in untreated (FIG. 15Bb) neurons. The input-output curve produced from injecting step currents into recorded neurons was shifted leftwards after chronic exposure to Aβ (FIG. 15D). These results indicate that chronic treatment of cultured hippocampal neurons with Aβ₁₋₄₂ increases neuronal excitability.

### Example 23

### APP Transgenic AD Mice (3×2Tg) Exhibited Hyperexcitation

To test whether enhanced in vivo Aβ expression induces hippocampal hyperexcitation, field recordings in CA1 region of hippocampal slices prepared from 3×Tg-AD mice indicate that in 3×Tg mice (10 month-old), the input-output curve shifted leftward (FIG. 16A) and carbachol (CCh)-induced much stronger network synchronization (FIG. 16B) compared to age-matched WT mice (Oddo et al., 2003). The frequency and bursting cluster numbers of θ oscillations were significantly different between AD and WT slices. These results show that the hippocampal neurons/circuits in 10-month-old 3×Tg-AD mice exhibit hyperexcitation and are more susceptible to CCh-induced network synchronization. To test whether 3×TgAD mice exhibit hyperexcitation, EEG activity from two 3×TgAPP mice and two age-matched (18-month-old) WT mice, showed seizure activity (FIG. 17A) in 3×TgAPP (n=2) but not WT (n=2) mice (FIG. 17B), which shows that 3×TgAD mice exhibit epileptic seizure-like EEG activity.

### Example 24

### Roles of α7-nAChR in Aβ-Induced Neuronal Hyperexcitation in Cultured Hippocampal Neurons

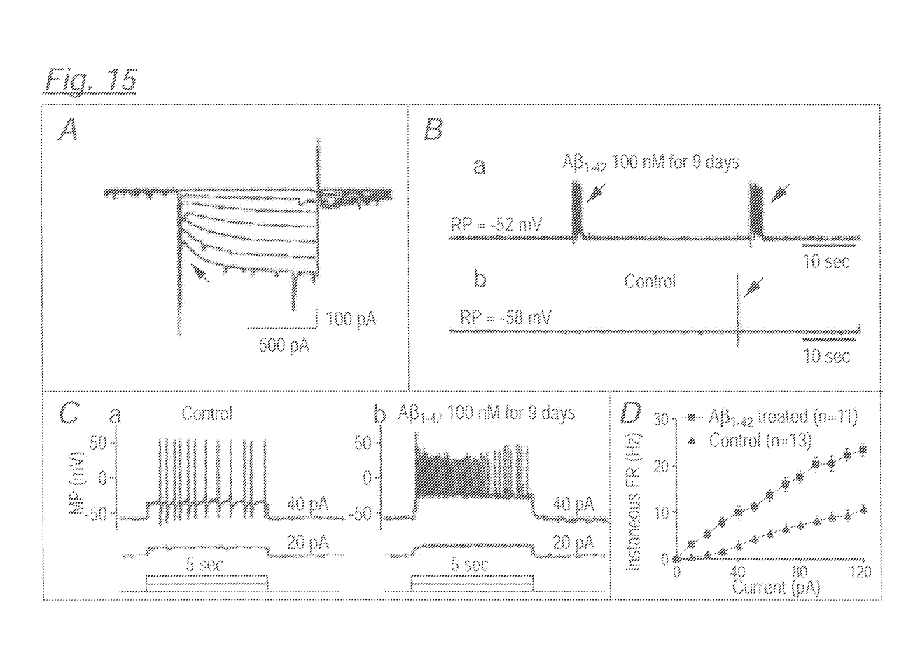

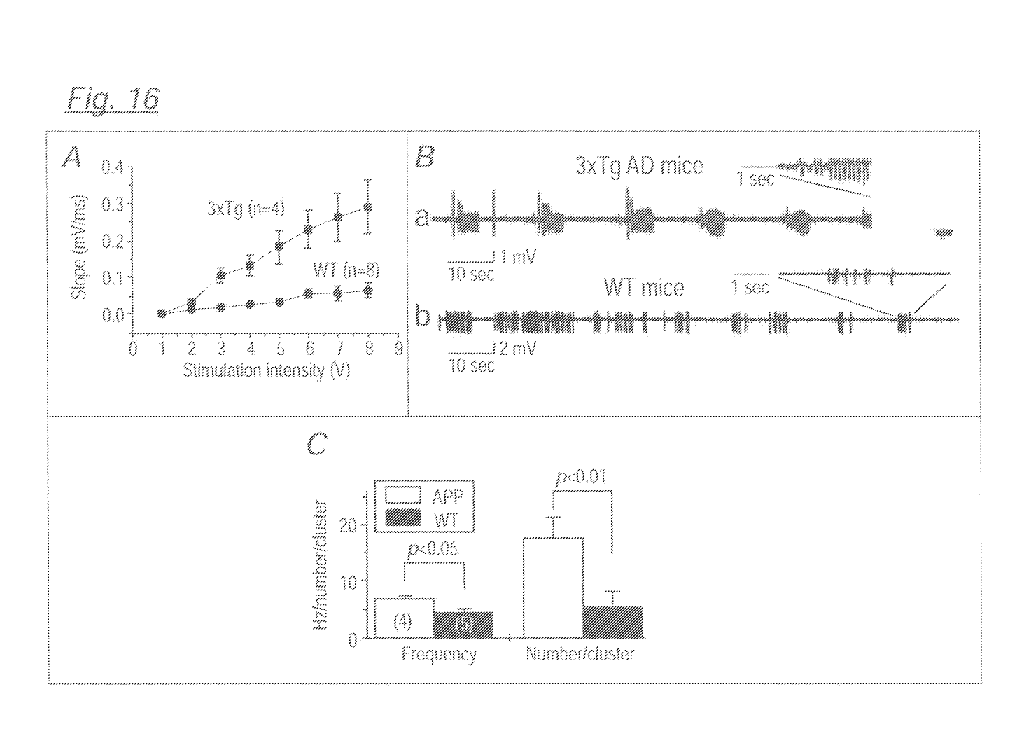

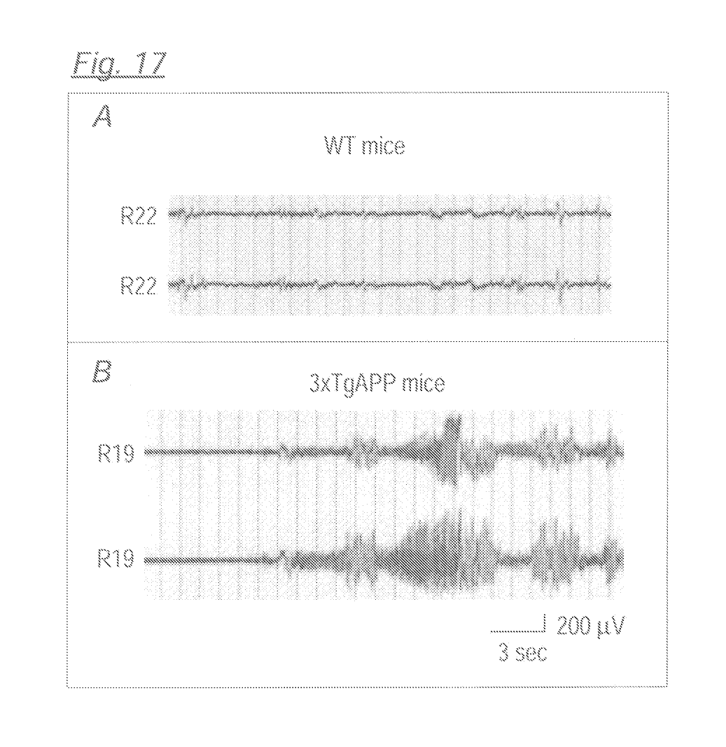

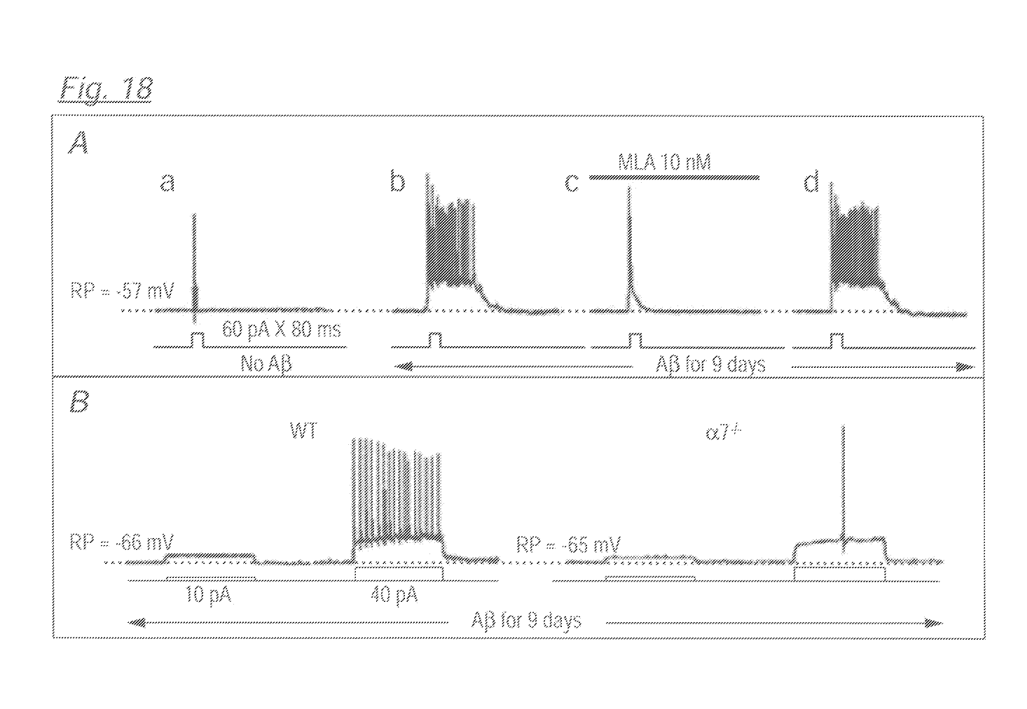

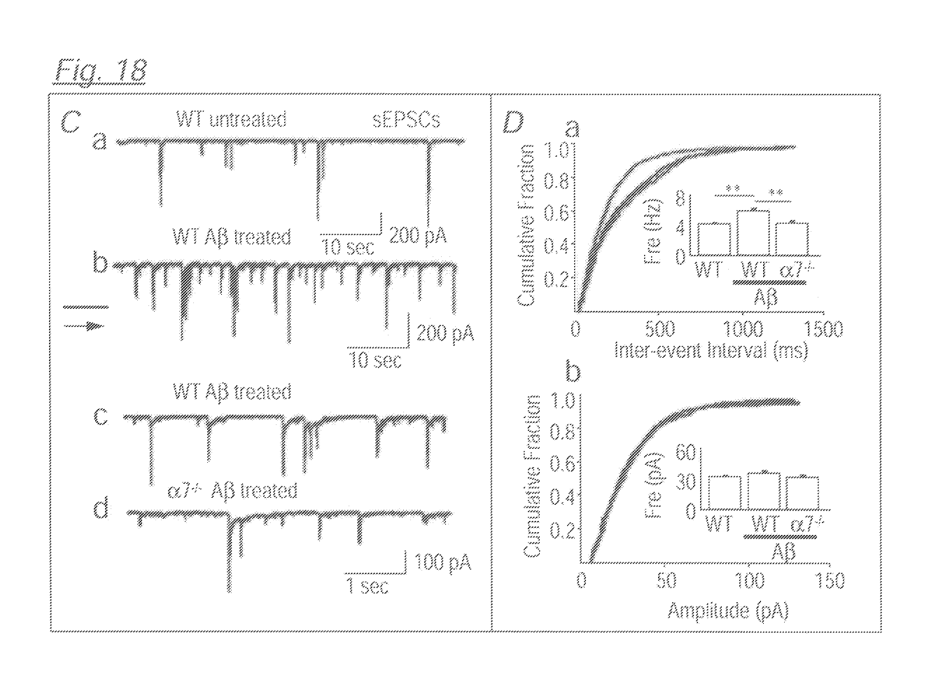

To further investigate whether pharmacological block of α7-nAChR eliminated the expression of neuronal hyperexcitation after chronic Aβ exposure. As shown in FIG. 18A, enhanced neuronal excitation (following a depolarizing pulse) was reversibly eliminated by a selective α7-nAChR antagonist, MLA (FIG. 18Ac). Comparing cultured neurons from WT and α7−/− mice could further establish if α7-nAChRs play a role in the induction of Aβ-induced neuronal hyperexcitation by comparing cultured neurons from WT and α7−/− mice. FIG. 17B shows that chronic exposure to Aβ failed to evoke neuronal hyperexcitation in the neurons from α7−/− mice. Finally, comparing sEPSCs in the neurons prepared from WT and α7−/− mice after exposure to Aβ showed that in WT mice, chronic Aβ enhances sEPSC frequency (FIG. 17Cb), but not amplitude (FIG. 18Db). Interestingly, α7-nAChR subunit gene deletion prevented Aβ's effect on sEPSCs (FIG. 18Cd,Da). These results show that α7-nAChRs play important roles in both induction and expression of neuronal hyperexcitation after chronic Aβ exposure and the enhancement of presynaptic α7-nAChR-mediated modulation of excitatory neurotransmission may contribute to Aβ-induced neuronal hyperexcitation.

### Example 24

### α7−/− Mice Exhibited Low Seizure Susceptibility

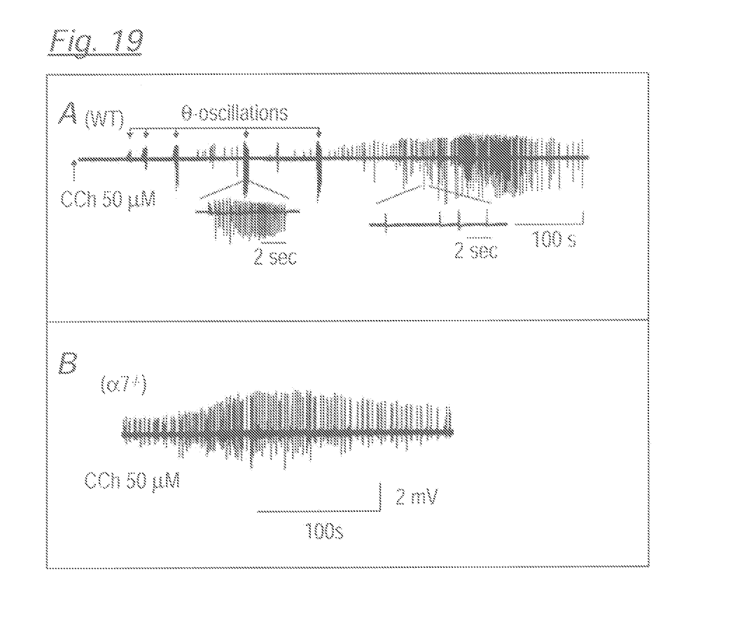

Comparing CCh-induced network synchronization in the hippocampal CA1 slices from WT and α7−/− mice (6 months old) using field potential recordings allowed testing of whether α7-nAChRs contribute to network synchronization and seizure susceptibility. FIG. 19 shows that in WT slices, bath-perfusion of 50 μM CCh induced two types of network synchronizations: single field bursting (similar to interictal) below 1 Hz, and clustering bursts in the range of 4-12 Hz (θ-oscillations, FIG. 19A, indicated by red arrows). In contrast, in α7−/− slices, CCh failed to induce θ-oscillations although it still induced interictal-like events (FIG. 19B). Since glutamatergic transmission contributes to the formation of these clustering bursts, this indicates that α7-nAChR-mediated increase (showed in FIG. 18Cb) of glutamatergic synaptic transmission may be important in hippocampal network synchronizations (Crews and Masliah, 2010).

### Example 24

### α7−/− Hippocampal Neurons Exhibit Little Aβ Toxicity

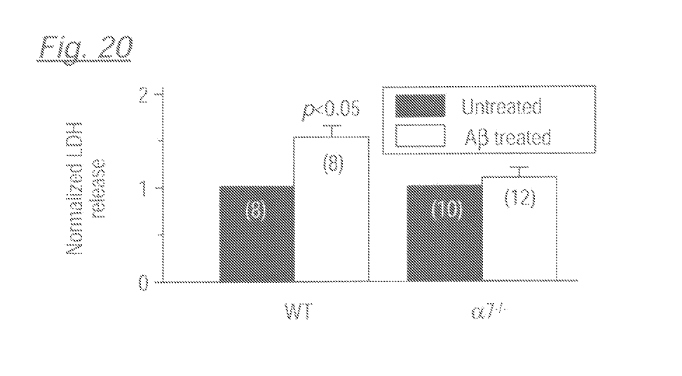

To test the roles played by α7-nAChRs in Aβ toxicity, the inventors treated α7−/− hippocampal neurons with Aβ₁₋₄₂, and found that Aβ did not show toxic effects on these α7−/− hippocampal neurons compared to WT hippocampal neurons (FIG. 20). These results demonstrate that α7-nAChRs play a key role in Aβ toxicity under our experimental conditions.

### Example 25

### Pathological Levels of Aβ Induce Hippocampal Neuron Toxicity

Systematically examining the effects of various Aβ exposure conditions (different concentrations, forms, exposure regimens and time courses) on neuronal toxicity, as measured by neuronal apoptosis, degeneration or death in primary culture neurons, demonstrates the effect that pathological levels of Aβ have in terms neurotoxicity (Sakono and Zako, 2010; Kitamura and Kubota, 2010; Crews and Masilah, 2010). Comparing cell loss and synaptic plasticity (LTP) between AD model and wild type (WT) mice provides a detailed description of Aβ toxicity under in vitro conditions. Determining hippocampal neuron loss in adult (>10 month-old) 3×Tg-AD mice can be shown using multiple approaches with brains of age-matched WT mice serving as positive controls. TUNEL-YOYO staining allows identification and staining of TUNEL-positive neurons in sections of the hippocampus prepared from 3×Tg-AD and WT mice (Resendes et al., 2004). On the same sections, the compact nuclei identified by TUNEL, also will be stained with the DNA binding cyanine dye YOYO-1. The condensed nuclear chromatin pattern associated with apoptosis in these cells can be be shown. Additional histological evidence of nuclear condensation in the hippocampal tissue can be shown using Nissl staining. Probing for the activation of caspases in degenerating neurons can be done using caspase-3 immunolabeling a recognizing the activated form of caspase-3, a biological change associated with apoptopic cell death (Resendes, et al., 2004)

### Example 25

### To Characterize Aβ-Induced Cytotoxicity in Cultured Hippocampal Neurons

Primary cultures of rat hippocampal neurons can also be used to characterize the toxic effect of Aβ, this includes the of use electrophysiological recordings under Aβ treated (Aβ₁₋₄₂, 100 nM for 10 days) and untreated conditions, and hippocampal neurons' viability can be assessed using MTT assay (Agostinho and Oliveria, 2003). Apoptosis of cultured hippocampal neurons in Aβ treated and untreated neurons using the same experimental approaches as described above can serve as a model for neuronal degeneration. Characterizing the neurotoxic effect of Aβ in primary cultures of hippocampal neurons by manipulating the protocol of Aβ treatment can establish the effects of Aβ on hippocampal neuron viability (MTT assay) under different Aβ conditions including different Aβ concentrations (from 0.1 to 1,000 nM), Aβ formats (monomers, oligomers or fibrils) and Aβ treatment lengths (1-15 days).

### Example 26

### Effects of Endogenous and Exogenous Aβ on Hippocampal Synaptic Plasticity

In other experiments, the effects of endogenous and exogenous Aβ on hippocampal synaptic plasticity can be measured by analyzing hippocampal slices from LTP between 3×Tg AD and WT mice and further testing the effects of exogenous Aβ on hippocampal Shaffer collateral-CA1 LTP by bath-perfusion of Aβ to hippocampal slices as previously described (Yang et al., 2008; Vitolo et al., 2002). Modulating LTP by induced by different protocols (high frequency, theta burst or weak presynaptic stimulation) allows examination of the effects of different Aβ conditions (concentrations, Aβ formats and Aβ treatment times) on LTP induction and maintenance.

### Example 27

### Mechanisms of Aβ-Induced Neural Toxicity

As described herein, Aβ exhibits extremely high affinity binding to α7-nAChRs and modulates α7-nAChR function (Wang et al., 2000a; Wang et al., 2000b; Liu et al., 2009; Liu et al., 2001; Pettit et al., 2001; Wu et al., 2004a). In AD patient and animal models, there are significantly enhanced levels of nAChR α7 subunit expression (Jones et al., 2006; Counts et al., 2007b; Hellstron-Lindhal 2004a; Hellstron-Lindhal 2004b; Hellstron-Lindhal 1999; Dinley et al., 2002; Chu et al., 2005; Teaktong et al., 2004; Ikonomovic et al., 2009). Chronic exposure to Aβ up-regulates α7-nAChR expression in glial cells (Xiu et al., 2005; Yu et al., 2005). These indicates that Aβ upregulates α7-nAChR expression and function, which may be an important mechanism in Aβ toxicity. In addition, it has been well established that acute exposure to Aβ suppresses α7-nAChR function in a variety of preparations (Liu et al., 2009; Liu et al., 2001; Pettit et al., 2001; Wu et al., 2004a; Wu et al., 2004c). This acute inhibition may trigger longer-term α7-nAChR up-regulation (Govind et al., 2009). Without being bound by any particular theory, the inventors reason that α7-nAChRs are up-regulated by chronic exposure to Aβ both in cultured hippocampal neurons, and in APP AD model mice.

Further measurement of nAChR α7 subunit mRNA (qRT-PCR) and protein ([125I]α-Bgt binding) expression in cultured hippocampal neurons treated and untreated with Aβ can establish whether chronic exposure Aβ up-regulates α7-nACRs. (Liu et al., 2009; Wu, et al., 2004a; Yang et al., 2009) The use of the patch-clamp technique as previously reported can identify functional alterations of hippocampal α7-nAChRs (Liu et al., 2009; Wu at al., 2004a; Wu et al., 2004b; Zhao et al., 2003). This can further be applied to examine nAChR α7 subunit expression (mRNA and protein) in hAPP (hAPPJ20 and 3×Tg AD) and WT mice. For in vivo studies, the hAPPJ20 (Jackson Lab) mouse model demonstrates progressive neuronal hyperexcitation and epileptic seizures 16 and triple-transgenic mouse model (3×Tg-AD) harboring PSI (M146V), APP (Swe), and tau (P301L) transgenes, allows observation of the influence of combined genetic factors on AD-like phenotypes (Oddo et al., 2003). Examples include an age-dependent increase in tau expression with tau expression levels playing an important role in determining neuronal excitability and synaptic dysfunction (Oddo et al., 2003; Roberson et al., 2007). Hippocampal and whole brain tissues collected for qRT-PCR and [125I]α-Bgt binding experiments can be collected from hAPP and WT mice. Testing both nAChR α7 subunit expression and Aβ₁₋₄₂ levels (ELISA) at different ages of AD mice (e.g., 3, 6, 10 and 18 months) and comparing these with age-matched WT (control) mice can determine the relationship of α7-nAChR expression and Aβ deposition.

To further identify whether chronic Aβ upregulates presynaptic or postsynaptic α7-nAChRs in cultured hippocampal neurons, patch-clamp whole-cell recording techniques can measure somatodendritic whole cell currents induced by α7-nAChR agonists for comparison of the currents between Aβ treated and untreated neurons. Measurement of spontaneous excitatory postsynaptic currents (sEPSCs), and comparing these currents between Aβ treated and untreated neurons allows monitoring of the functional changes of presynaptic α7-nAChRs and Aβ treatment increases in sEPSCs (frequency) can further be measured in cultured neurons prepared from α7−/− mice. An increase of both presynaptic and postsynaptic α7-nAChR function is shown (FIG. 14) and may further support data showing that Aβ likely up-regulates α7-nAChRs through a posttranslational mechanism (FIG. 14). To avoid pleiotropic effects of Aβ, where alterations occur in other ion channel and synaptic function in cultured neurons beyond α7-nAChRs, other cell types such as SH-SY5Y cells or heterologously expressed α7-nAChRs in the SH-EP 1 cell line can serve as controls (Zhao et al., 2003). Different Aβ conditions that exhibit toxic (e.g., 100 nM, oligomers for 10 days) or nontoxic effect (e.g., 100 nM monomers for 10 days) can evaluate the relationship of α7-nAChR upregulation and AB toxicity. Finally, electrophysiological recordings in primary cultured hippocampal neurons (patch-clamp), in hippocampal slices (field recording) and in living mice (EEG) can demonstate how α7-nAChRs mediate Aβ-induced hyperexcitation to confirm existing reports that in AD patients and model animals, Aβ and α7-nAChRs express at an aberrantly high level, and neuronal circuits exhibit hyperexcitation (Jones et al., 2006; Palop et al., 2007; Palop et al., 2009; Hellstron-Lindhal 2004a; Hellstron-Lindhal 2004b; Spencer et al., 2006; Westmark et al., 2008; Busche et al., 2008). The contribution of α7-nAChRs to modulation of neuronal excitability and the generation of epileptic seizures indicate that upregulated α7-nAChRs by Aβ may contribute to neural hyperexcitation (Damaj et al., 1999; Caroll et al., 2007; Miner and Collns, 1989; Miner, Marks, and Collins, 1986).

### Example 28

### The Roles of α7-nAChRs in Aβ-Induced Neural Hyperexcitation

α7-nAChRs exhibit high Ca2+ permeability, and activation of α7-nAChRs increases intracellular calcium levels, which suggests that Aβ-induced increase in neuronal intrinsic excitability is mediated through α7-nAChRs (Castro and Albuquerque, 1995; Delbono et al., 1997). Chronic exposure of cultured neurons to Aβ elevates intracellular Ca2+ levels. Establishing whether this effect is mediated through nAChRs, a comparison of intracellular Ca2+ levels between Aβ treated (e.g., 100 nM, oligomers for 10 days) and un-treated hippocampal neuron cultures using Fura-2 Ca2+ imaging, and can determine the roles of α7-nAChRs in Aβ-induced increases of intrinsic excitability (Wu et al., 2006; Misaki et al., 2007; Wu et al., 2009). Furthermore, comparing neuronal excitability (patch-clamp) and intracellular Ca2+-levels (fura-2) in Aβ treated hippocampal neurons prepared from α7−/− and WT mice can further confirm that after chronic treatment with Aβ, up-regulated α7-nAChRs will elevate intracellular Ca 2+ concentrations and is related to increased neuronal excitability. Furthermore, α7-nAChRs may also contribute to chronic Aβ-induced increases neuronal hyperexcitation through a synaptic mechanism. Without being bound by any particular theory, because Aβ acts on presynaptic α7-nAChRs and elevates intracellular Ca2+ levels, which can promote neurotransmitter (mainly glutamate) release, Aβ possibly induces neuronal hyperexcitation through this mechanism, particularly if α7-nAChRs have been up-regulated (Dougherty, Wu, and Nichols, 2003). To test this possibility, the frequency and amplitude of sEPSCs can be analyzed and compared between Aβ (100 nM, oligomers for 10 days) treated neurons prepared from α7−/− and WT mice to determine whether Aβ-induced alterations of sEPSCs are mediated through a presynaptic mechanism. Also, miniature EPSCs (mEPSCs, in the presence of 1 μM TTX) can be compared between α7−/− and WT mice after Aβ treatment in cultured hippocampal neurons. This allows determination of the roles of α7-nAChRs in Aβ-induced initiation of neural hyperexcitation (e.g., in α7−/− hippocampal neurons, Aβ is not able to induced neural hyperexcitation). Furthermore, the effects of α7-nAChR antagonists (MLA 10 nM or α-bungarotoxin 100 nM) on Aβ-induced neural hyperexcitation can establish whether α7-nAChRs also play a role in Aβ-induced expression of neural hyperexcitation.

Importantly, the specificity of α7-nAChR as a target for Aβ-induced neuronal hyperexcitation is to be addressed since other evidence indicates that Aβ exhibits quite broad effects on a variety of receptors/channels under in vitro experimental conditions (Demuro, Parker and Stutzmnann, 2010; Chen and Yan, 2010; Ondrejack et al., 2010). However, most acute effects of Aβ on these receptors/channels either on astrocytes or on neurons require much higher concentrations of Aβ (micro-molar level) than those seen in AD patient brain (low nano-molar level) (Abramov, Canevari and Duchen, 2004; Abramov and Duchen, 2005; Case et al, 2009; Cirrito and Holtzmann, 2003). Thus, the concentrations of Aβ at pathological levels (e.g., 1-100 nM, oligomers for acute exposure or chronic exposure for 10 days), might specifically act on α7-nAChRs to affect neuronal excitability. Measurement of acute or chronic effects of Aβ on various voltage-gated (Na+, K+ and C2+) and ligand-gated ion channels (e.g., ionotropic glutamate receptors, GABA A receptor) can show whether α7-nAChR is a specific target to mediate Aβ, if Aβ fails to affect these ion channel- or receptor-mediated currents but selectively affects α7-nAChR function.

It is important to further understand whether α7-nAChRs contribute to neuronal network hyperexcitation/synchronization. Measurement of neuronal network activity using field-recording technique in hippocampal slices (450 μm) prepared from adult or aged mice can be coupled with chemical induction (e.g., CCh 50 μM or 4-AP 50 μM) or tetanic stimulation as previously reported (Song et al., 2005). Comparing the neuronal hyperexcitation/synchronization in different types of mice, such as variable age-groups (3 and 12 month-old) WT, APP transgenic (3×Tg APP or J20 APP), nAChR α7−/− and APPα7−/− mice can assess the α7-nAChRs contribution to neuronal network hyperexcitation/synchronization. Measurement of brain EEG activity in free-moving mice can determined whether α7-nAChRs contribute to epileptogenesis in APP AD mice and after first measurement of animal EEG activity, tests of neuronal network activity in hippocampal slices can assess the α7-nAChRs contribution to neuronal network hyperexcitation/synchronization as a model for epileptogenesis in APP AD.

### Example 2

### Evaluating the Roles of α7-nAChRs in Aβ-Induced Neural Toxicity

In AD patients and AD model animals, α7-nAChRs express at an aberrantly high level and the enhanced α7-nAChRs on glutamatergic synaptic terminals could trigger more glutamate release and result in excitatory toxicity (Counts et al., 2007b; Ikonomovic et al., 2009; An et al., 2010; Mousavi and Nordberg, 2006). Because α7-nAChRs exhibit extremely high permeability to Ca2+ and enhanced α7-nAChRs on somatodendratic area of cells could induce intracellular Ca2+ overload, neurodegeneration could be trigged and amplified by contribution of α7-nAChRs to the modulation of neuronal excitability and the generation of epileptic seizures (Couturier et al., 1990; Bertrand, et al., 1992; Damaj et al., 1999; Caroll et al., 2007; Miner and Collins, 1989; Miner, Marks, and Collins, 1986). It is important to determine whether Aβ-induced neurotoxicity is mediated through α7-nAChRs. By eliminating α7-nAChR function, one can compare toxic effects (e.g., HDL release) after chronic Aβ treatment on cultured hippocampal neurons between WT and nAChR α7−/− mice, and also compare Aβ toxicity between hippocampal culture neurons prepared from WT mice that are present or absent α7-nAChR antagonist (e.g., MLA 1-10 nM or α-bungarotoxin 10-100 nM) during Aβ treatment. In contrast, an enhancement of α7-nAChR function can be achieved using an α7-nAChR positive allosteric modulator (PNU-120596, 100 nM) during Aβ treatment to test Aβ toxicity in different experimental groups, such as control (Aβ untreated), Aβ treated, Aβ and PNU-120596 co-treated, and PNU-120596 treated. Various forms of Aβ that do not exhibit or have only mild toxic effect on hippocampal neurons (e.g., with low Aβ concentrations or shorter Aβ treatment period) can be used to gauge the magnitude of α7-nAChR contribution to Aβ neurotoxicity since PNU-120596 itself may not induce cytotoxicity and may increase Aβ toxicity (Hu, Gopalakirshnan and Li, 2009). Alternatively, α7-nAChRs may mediate Aβ toxicity through hyperexcitation. Eliminating neural hyperexcitation using the antagonists of ionotropic glutamate receptors (NBQX 10 μM or MK801 20 μM) during Aβ treatment, and then testing Aβ toxicity can shed light on this question. In addition, enhancement of neural hyperexcitation to mimic Aβ toxicity with and without α7-nAChRs, can identify whether α7-nAChRs mediate Aβ toxicity occurs through hyperexcitation. Alternatively, a K+ channel blocker, 4-aminopyradine (4-AP 100 μM) or glutamate (5 mM) to treat hippocampal culture neurons, with tests for neurotoxicity or comparisons of this excitatory toxicity between WT and nAChR α7−/− mice, can be applied. Deficits of synaptic plasticity in hippocampal CA1 region in AD model animals due to α7-nAChRs can be identified by comparing WT hippocampal Schafer collateral-CA1 LTP between Aβ treated (e.g., 200 nM, oligomers, acute perfusion to hippocampal slice or pre-incubation with Aβ for 1-3 hrs) and untreated slices. Alternatively, comparisons can be made among hippocampal LTP between the hippocampal slices prepared from APP AD mice with and without α7-nAChRs. For in vivo studies, the loss of hippocampal neurons in APP mice with and without α7-nAChRs can be measured. Mating together APP transgenic (J20) and α7−/− mice to generate APP/AD α7−/− mice can be used for further observation of the influence of combined genetic factors on AD-like phenotypes. Different age-groups (3 and 12 months) WT, APP transgenic (3×Tg APP or J20 APP), nAChR α7−/− and APPα7−/− mice can be used for these experiments.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are methods of prognosing, diagnosing, treating, and/or other various diseases and conditions as related to NAChRs and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

in closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

Abranov, A. Y., Canevari, L., Duchen, M. R. Calcium signals induced by amyloid beta peptide and their consequences in neurons and astrocytes in culture. Biochim Biophys Acta 1742, 81-87 (2004).
Abramov, A. Y., Duchen, M. R. The role of an astrocytic NADPH oxidase in the neurotoxicity of amyloid beta peptides. Philos Trans R Soc Loud B Biol Sci 360, 2309-2314 (2005).
Agostinho, P., Oliveira, C. R. Involvement of calcineurin in the neurotoxic effects induced by amyloid-beta and prion peptides. Eur J Neurosci 17, 1189-1196 (2003).
Alkondon, M., Albuquerque, E. X. Diversity of nicotinic acetylcholine receptors in rat hippocampal neurons. Ill. Agonist actions of the novel alkaloid epibatidine and analysis of type II current. J Pharmacol Exp Ther 274, 771-782 (1995).
Alkondon, M. A. Single in vivo application of cholinesterase inhibitors has neuron type-specific effects on nicotinic receptor activity in guinea pig hippocampus. Journal of Pharmacology and Experimental Therapeutics 328, 69-82 (2008).
Amatniek, J. C., et al. Incidence and predictors of seizures in patients with Alzheimer's disease. Epilepsia 47, 867-872 (2006).
An, Y., et al. Amyloid precursor protein gene mutated at Swedish 670/671 sites in vitro induces changed expression of nicotinic acetylcholine receptors and neurotoxicity. Neurochem Int 57, 647-654 (2010).
Aracava, Y., et al. Memantine blocks alpha7*nicotinic acetylcholine receptors more potently than n-methyl-D-aspartate receptors in rat hippocampal neurons. J Pharmacol Exp Ther 312, 1195-1205 (2005).
Arundine, M., Tymianski, M. Molecular mechanisms of calcium-dependent neurodegeneration in excitotoxicity. Cell Calcium 34, 325-337 (2003).
Azam L., Winzer-Serhan U., Leslie F. M. Co-expression of alpha7 and beta2 nicotinic acetylcholine receptor subunit mRNAs within rat brain cholinergic neurons. Neuroscience 119, 965-977 (2003).
Bencherif, M., Schmitt, J. D. Targeting neuronal nicotinic receptors: a path to new therapies. Curr Drug Targets CNS Neurol Disord 1, 349-357 (2002).
Bertrand, D., Bertrand, S., Ballivet, M. Pharmacological properties of the homomeric alpha 7 receptor. Neurosci Lett 146, 87-90 (1992).
Biton, B., et al. SSR180711, a novel selective alpha7 nicotinic receptor partial agonist: (1) binding and functional profile. Neuropsychopharmacology 32, 1-16 (2007).
Broide, R. S., et al. Increased sensitivity to nicotine-induced seizures in mice expressing the L250T alpha 7 nicotinic acetylcholine receptor mutation. Mol Pharmacol 61, 695-705 (2002).
Buccafusco, J. J., et al. Long-lasting cognitive improvement with nicotinic receptor agonists: mechanisms of pharmacokineticpharmacodynamic discordance. Trends Pharmacol Sci 26, 352-360 (2005).
Buckingham, S. D., et al. Nicotinic acetylcholine receptor signalling: roles in Alzheimer's disease and amyloid neuroprotection. Pharmacol Rev 61, 39-61 (2009).
Burghaus L., et al. Quantitative assessment of nicotinic acetylcholine receptor proteins in the cerebral cortex of Alzheimer patients. Brain Res Mol Brain Res 76, 385-388 (2000).
Busche, M. A., et al. Clusters of hyperactive neurons near amyloid plaques in a mouse model of Alzheimer's disease. Science 321, 1686-1689 (2008).
Casley, C. S., et al. Up-regulation of astrocyte metabotropic glutamate receptor 5 by amyloidbeta peptide. Brain Res (2009).
Castro, N. G., Albuquerque, E. X. alpha-Bungarotoxin-sensitive hippocampal nicotinic receptor channel has a high calcium permeability. Biophys J 68, 516-524 (1995).
Chen, J. X., Yan, S. S. Role of mitochondrial amyloid-beta in Alzheimer's disease. J Alzheimers Dis 20 Suppl 2, S569-578 (2010).
Chu, L. W., et al. Increased alpha 7 nicotinic acetylcholine receptor protein levels in Alzheimer's disease patients. Dement Geriatr Cogn Disord 19, 106-112 (2005).
Cirrito, J. R., Holtzman, D. M. Amyloid beta and Alzheimer disease therapeutics: the devil may be in the details. J Clin Invest 112, 321-323 (2003).
Clarke, P. B., Reuben, M., el-Bizri, H. Blockade of nicotinic responses by physostigmine, tacrine and other cholinesterase inhibitors in rat striatum. Br J Pharmacol 111, 695-702 (1994).
Counts, S. E., et al. Alpha7 nicotinic receptor up-regulation in cholinergic basal forebrain neurons in Alzheimer disease. Arch Neurol 64, 1771-1776 (2007a).
Counts S. E., et al. Alpha7 nicotinic receptor up-regulation in cholinergic basal forebrain neurons in Alzheimer disease. Arch Neurol 64:1771-1776 (2007b).
Couturier, S., et al. A neuronal nicotinic acetylcholine receptor subunit (alpha 7) is developmentally regulated and forms a homo-oligomeric channel blocked by alpha-BTX. Neuron 5, 847-856 (1990).
Crews, L., Masliah, E. Molecular mechanisms of neurodegeneration in Alzheimer's disease. Hum Mol Genet 19, R12-20 (2010).
D'Andrea, M. R., Nagele, R. G. Targeting the alpha 7 nicotinic acetylcholine receptor to reduce amyloid accumulation in Alzheimer's disease pyramidal neurons. Curr Pharm Des 12, 677-684 (2006).
Damaj, M. I., et al. Pharmacological characterization of nicotine-induced seizures in mice. J Pharmacol Exp Ther 291, 1284-1291 (1999).
Del Vecchio, R. A., et al. Increased seizure threshold and severity in young transgenic CRND8 mice. Neurosci Lett 367, 164-167 (2004).
Delbono, O., et al. Activation of the recombinant human alpha 7 nicotinic acetylcholine receptor significantly raises intracellular free calcium. J Pharmacol Exp Ther 280, 428-438 (1997).
Demuro, A., Parker, I., Stutzmann, G. E. Calcium signaling and amyloid toxicity in Alzheimer disease. J Biol Chem 285, 12463-12468 (2010).
Dineley K. T., et al. beta-Amyloid peptide activates alpha 7 nicotinic acetylcholine receptors expressed in Xenopus oocytes. J Biol Chem 277, 25056-25061 (2002a).
Dineley K. T., et al. Accelerated plaque accumulation, associative learning deficits, and up-regulation of alpha 7 nicotinic receptor protein in transgenic mice co-expressing mutant human presenilin 1 and amyloid precursor proteins. J Biol Chem 277, 22768-22780 (2002b).
Dolezal, V., Kasparova, J. Beta-amyloid and cholinergic neurons. Neurochem Res 28, 499-506 (2003).
Dougherty, J. J., Wu, J., Nichols, R. A. Beta-amyloid regulation of presynaptic nicotinic receptors in rat hippocampus and neocortex. J Neurosci 23, 6740-6747 (2003).
Dziewczapolski, G., et al. Deletion of the alpha 7 nicotinic acetylcholine receptor gene improves cognitive deficits and synaptic pathology in a mouse model of Alzheimer's disease. J Neurosci 29, 8805-8815 (2009).
El-Hajj R. A., McKay S. B., McKay D. B. Pharmacological and immunological identification of native alpha7 nicotinic receptors: evidence for homomeric and heteromeric alpha7 receptors. Life Sci 81, 1317-1322 (2007).
Flores C. M., et al. A subtype of nicotinic cholinergic receptor in rat brain is composed of alpha 4 and beta 2 subunits and is up-regulated by chronic nicotine treatment. Mol Pharmacol 41:31-37 (1992).
Fraser, S. P., Suh, Y. H., Djamgoz, M. B. Ionic effects of the Alzheimer's disease beta-amyloid precursor protein and its metabolic fragments. Trends Neurosci 20, 67-72 (1997).
Fu W., Jhamandas J. H. Beta-amyloid peptide activates non-alpha7 nicotinic acetylcholine receptors in rat basal forebrain neurons. J Neurophysiol 90, 3130-3136 (2003).
Geerts H. Indicators of neuroprotection with galantamine. Brain Res Bull 64, 519-524 (2005).
Gopalakrishnan M., et al. Stable expression, pharmacologic properties and regulation of the human neuronal nicotinic acetylcholine alpha 4 beta 2 receptor. J Pharmacol Exp Ther 276, 289-297 (1996).
Govind, A. P., Vezina, P., Green, W. N. Nicotine-induced upregulation of nicotinic receptors: underlying mechanisms and relevance to nicotine addiction. Biochem Pharmacol 78, 756-765 (2009).
Grassi F., et al. Amyloid beta(1-42) peptide alters the gating of human and mouse alpha-bungarotoxin-sensitive nicotinic receptors. J Physiol 547, 147-157 (2003).
Haass C., Selkoe D. J. Soluble protein oligomers in neurodegeneration: lessons from the Alzheimer's amyloid beta-peptide. Nat Rev Mol Cell Biol 8, 101-112 (2007).
Hellstrom-Lindahl, E., et al. A. Regional distribution of nicotinic receptor subunit mRNAs in human brain: comparison between Alzheimer and normal brain. Brain Res Mol Brain Res 66, 94-103 (1999).
Hellstrom-Lindahl, E., et al. Nicotine reduces A beta in the brain and cerebral vessels of APPsw mice. Eur J Neurosci 19, 2703-2710 (2004a).
Hellstrom-Lindahl, E., et al. A. Reduced levels of Abeta 40 and Abeta 42 in brains of smoking controls and Alzheimer's patients. Neurobiol Dis 15, 351-360 (2004b).
Henderson Z., et al. Somato-dendritic nicotinic receptor responses recorded in vitro from the medial septal diagonal band complex of the rodent. J Physiol 562, 165-182 (2005).
Hernandez, C. M., et al. Loss of alpha7 nicotinic receptors enhances beta-amyloid oligomer accumulation, exacerbating early-stage cognitive decline and septohippocampal pathology in a mouse model of Alzheimer's disease. J Neurosci 30, 2442-2453 (2010).
Horn R., Marty A. Muscarinic activation of ionic currents measured by a new whole-cell recording method. J Gen Physiol 92:145-159 (1988).
Hsia, A. Y., et al. Plaque-independent disruption of neural circuits in Alzheimer's disease mouse models. Proc Natl Acad Sci USA 96, 3228-3233 (1999).
Hu, M., Gopalakrishnan, M., Li, J. Positive allosteric modulation of alpha7 neuronal nicotinic acetylcholine receptors: lack of cytotoxicity in PC 12 cells and rat primary cortical neurons. Br J Pharmacol 158, 1857-1864 (2009).
Ikonomovic, M. D., et al. Cortical alpha7 nicotinic acetylcholine receptor and beta-amyloid levels in early Alzheimer disease. Arch Neurol 66, 646-651 (2009).
Ivy Carroll, F., et al. Synthesis, nicotinic acetylcholine receptor binding, antinociceptive and seizure properties of methyllycaconitine analogs. Bioorg Med Chem 15, 678-685 (2007).
Jensen A. A., et al. Neuronal nicotinic acetylcholine receptors: structural revelations, target identifications, and therapeutic inspirations. J Med Chem 48, 4705-4745 (2005).
Jones, I. W., et al. Alpha7 nicotinic acetylcholine receptor expression in Alzheimer's disease: receptor densities in brain regions of the APP(SWE) mouse model and in human peripheral blood lymphocytes. J Mol Neurosci 30, 83-84 (2006).
Kasa, P., Rakonczay, Z., Gulya, K. The cholinergic system in Alzheimer's disease. Prog Neurobiol 52, 511-535 (1997).
Khiroug S. S., et al. Rat nicotinic ACh receptor alpha7 and beta2 subunits co-assemble to form functional heteromeric nicotinic receptor channels. J Physiol 540:425-434 (2002).
Kitamura, A., Kubota, H. Amyloid oligomers: dynamics and toxicity in the cytosol and nucleus. FEBS J 277, 1369-1379 (2010).
Klein, W. L. Abeta toxicity in Alzheimer's disease: globular oligomers (ADDLs) as new vaccine and drug targets. Neurochem Int 41, 345-352 (2002).
Kuo Y. M., et al. Elevated abeta42 in skeletal muscle of Alzheimer disease patients suggests peripheral alterations of AbetaPP metabolism. Am J Pathol 156, 797-805 (2000).
Lahiri D. K., et al. Nicotine reduces the secretion of Alzheimer's beta-amyloid precursor protein containing beta-amyloid peptide in the rat without altering synaptic proteins. Ann N Y Acad Sci 965, 364-372 (2002).
Lamb P. W., Melton M. A., Yakel J. L. Inhibition of neuronal nicotinic acetylcholine receptor channels expressed in Xenopus oocytes by beta-amyloid1-42 peptide. J Mol Neurosci 27, 13-21 (2005).
Leiser, S. C., et al. A cog in cognition: how the alpha 7 nicotinic acetylcholine receptor is geared towards improving cognitive deficits. Pharmacol Ther 122, 302-311 (2009).
Levin, E. D., Rezvani, A. H. Nicotinic treatment for cognitive dysfunction. Curr Drug Targets CNS Neurol Disord 1, 423-431 (2002).
Leonard, A. S., McNamara, J. O. Does epileptiform activity contribute to cognitive impairment in Alzheimer's disease? Neuron 55, 677-678 (2007).
Li, X. D., Buccafusco, J. J. Effect of beta-amyloid peptide 1-42 on the cytoprotective action mediated by alpha7 nicotinic acetylcholine receptors in growth factor-deprived differentiated PC-12 cells. J Pharmacol Exp Ther 307, 670-675 (2003).
Lindstrom J. Neuronal nicotinic acetylcholine receptors. Ion Channels 4:377-450 (1996a).
Lindstrom J., et al. Structure and function of neuronal nicotinic acetylcholine receptors. Prog Brain Res 109, 125-137 (1996b).
Liu, Q., Kawai, H., Berg, D. K. beta-Amyloid peptide blocks the response of alpha 7-containing nicotinic receptors on hippocampal neurons. Proc Natl Acad Sci USA 98, 4734-4739 (2001).
Liu Q., et al. Dissecting the signaling pathway of nicotine-mediated neuroprotection in a mouse Alzheimer disease model. FASEB J 21, 61-73 (2007a).
Liu, Q., et al. A novel nicotinic acetylcholine receptor subtype in basal forebrain cholinergic neurons with high sensitivity to amyloid peptides. J Neurosci 29, 918-929 (2009).
Liu Z., Zhang J., Berg D. K. Role of endogenous nicotinic signaling in guiding neuronal development. Biochem Pharmacol 74, 1112-119 (2007b).
Lopes, C., et al. Competitive antagonism between the nicotinic allosteric potentiating ligand galantamine and kynurenic acid at alpha7* nicotinic receptors. J Pharmacol Exp Ther 322, 48-58 (2007).
Lopez-Hernandez, G., et al. Partial agonist and neuromodulatory activity of S 24795 for alpha7 nAChR responses of hippocampal interneurons. Neuropharmacology 53, 134-144 (2007).
Lukas R. J., et al. International Union of Pharmacology. Current status of the nomenclature for nicotinic acetylcholine receptors and their subunits. Pharmacol Rev 51:397-401 (1999).
Majd, S., et al. Different fibrillar Abeta 1-42 concentrations induce adult hippocampal neurons to reenter various phases of the cell cycle. Brain Res 1218, 224-229 (2008).
Martin, S. E., de Fiebre, N. E., de Fiebre, C. M. The alpha7 nicotinic acetylcholine receptorselective antagonist, methyllycaconitine, partially protects against beta-amyloid1-42 toxicity in primary neuron-enriched cultures. Brain Res 1022, 254-256 (2004).
Mansvelder H. D., et al. Nicotinic modulation of neuronal networks: from receptors to cognition. Psychopharmacology (Berl) 184, 292-305 (2006).
Mehta P. D., et al. Plasma and cerebrospinal fluid levels of amyloid beta proteins 1-40 and 1-42 in Alzheimer disease. Arch Neurol 57, 100-105 (2000).
Meldrum, B. S. Excitotoxicity and selective neuronal loss in epilepsy. Brain Pathol 3, 405-412 (1993).
Miner, L. L., et al. Genetic analysis of nicotine-induced seizures and hippocampal nicotinic receptors in the mouse. J Pharmacol Exp Ther 239, 853-860 (1986).
Miner, L. L., Collins, A. C. Strain comparison of nicotine-induced seizure sensitivity and nicotinic receptors. Pharmacol Biochem Behav 33, 469-475 (1989).
Minkeviciene, R., et al. Amyloid beta-induced neuronal hyperexcitability triggers progressive epilepsy. J Neurosci 29, 3453-3462 (2009).
Misaki, N., et al. Iptakalim, a vascular ATP-sensitive potassium (KATP) channel opener, closes rat pancreatic beta-cell KATP channels and increases insulin release. J Pharmacol Exp Ther 322, 871-878 (2007).
Molinari, E. J., et al. Up-regulation of human alpha7 nicotinic receptors by chronic treatment with activator and antagonist ligands. Eur J Pharmacol 347, 131-139 (1998).
Moser N., et al. Evaluating the suitability of nicotinic acetylcholine receptor antibodies for standard immunodetection procedures. J Neurochem 102, 479-492 (2007).
Mousavi, M., Nordberg, A. Expression of the alpha7, alpha4 and alpha3 nicotinic receptor subtype in the brain and adrenal medulla of transgenic mice carrying genes coding for human AChE and beta-amyloid. Int J Dev Neurosci 24, 269-273 (2006).
Mudo G., Belluardo N., Fuxe K. Nicotinic receptor agonists as neuroprotective/neurotrophic drugs. Progress in molecular mechanisms. J Neural Transm 114:135-147 (2007).
Nagele R. G., et al. Intracellular accumulation of beta-amyloid(1-42) in neurons is facilitated by the alpha 7 nicotinic acetylcholine receptor in Alzheimer's disease. Neuroscience 110, 199-211 (2002).
Nimmrich, V., Ebert, U. Is Alzheimer's disease a result of presynaptic failure? Synaptic dysfunctions induced by oligomeric beta-amyloid. Rev Neurosci 20, 1-12 (2009).
Nordberg, A. Neuroprotection in Alzheimer's disease—new strategies for treatment. Neurotox Res 2, 157-165 (2000).
Nordberg A. Nicotinic receptor abnormalities of Alzheimer's disease: therapeutic implications. Biol Psychiatry 49, 200-210 (2001).
Oddo, S., et al. Triple-transgenic model of Alzheimer's disease with plaques and tangles: intracellular Abeta and synaptic dysfunction. Neuron 39, 409-421 (2003).
Ondrejcak, T., et al. Alzheimer's disease amyloid beta-protein and synaptic function. Neuromolecular Med 12, 13-26 (2010).
Palop, J. J., et al. Aberrant excitatory neuronal activity and compensatory remodeling of inhibitory hippocampal circuits in mouse models of Alzheimer's disease. Neuron 55, 697-711 (2007).
Palop, J. J., Mucke, L. Epilepsy and cognitive impairments in Alzheimer disease. Arch Neurol 66, 435-440 (2009a).
Palop, J. J., Mucke, L. Synaptic Depression and Aberrant Excitatory Network Activity in Alzheimer's Disease: Two Faces of the Same Coin? Neuromolecular Med (2009b).
Pettit, D. L., Shao, Z., Yakel, J. L. beta-Amyloid(1-42) peptide directly modulates nicotinic receptors in the rat hippocampal slice. J Neurosci 21, RC120 2001).
Pym L., et al. Subtype-specific actions of beta-amyloid peptides on recombinant human neuronal nicotinic acetylcholine receptors (alpha7, alpha4beta2, alpha3beta4) expressed in Xenopus laevis oocytes. Br J Pharmacol 146, 964-971 (2005).
Ren, K., et al. The alpha7 nicotinic receptor agonist 4OH-GTS-21 protects axotomized septohippocampal cholinergic neurons in wild type but not amyloid-overexpressing transgenic mice. Neuroscience 148, 230-237 (2007).
Resendes, A. R., et al. Apoptosis in normal lymphoid organs from healthy normal, conventional pigs at different ages detected by TUNEL and cleaved caspase-3 immunohistochemistry in paraffin-embedded tissues. Vet Immunol Immunopathol 99, 203-213 (2004).
Rezvani, A. H., et al. Effect of R3487/MEM3454, a novel nicotinic alpha7 receptor partial agonist and 5-HT3 antagonist on sustained attention in rats. Prog Neuropsychopharmacol Biol Psychiatry 33, 269-275 (2009).
Roberson, E. D., et al. Reducing endogenous tau ameliorates amyloid beta-induced deficits in an Alzheimer's disease mouse model. Science 316, 750-754 (2007).
Sadot E., et al. Activation of m1 muscarinic acetylcholine receptor regulates tau phosphorylation in transfected PC12 cells. J Neurochem 66 877-880 (1996).
Sakono, M., Zako, T. Amyloid oligomers: formation and toxicity of Abeta oligomers. FEBS J 277, 1348-1358 (2010).
Saragoza P. A., et al. Identification of an alternatively processed nicotinic receptor alpha7 subunit RNA in mouse brain. Brain Res Mol Brain Res 117, 15-26 (2003).
Selkoe D. J. Translating cell biology into therapeutic advances in Alzheimer's disease. Nature 399, A23-31 (1999).
Severance E. G., Cuevas J. Distribution and synaptic localization of nicotinic acetylcholine receptors containing a novel alpha7 subunit isoform in embryonic rat cortical neurons. Neurosci Lett 372, 104-109 (2004a).
Severance E. G., et al. The alpha7 nicotinic acetylcholine receptor subunit exists in two isoforms that contribute to functional ligand-gated ion channels. Mol Pharmacol 66, 420-429 (2004b).
Sivaprakasam K. Towards a unifying hypothesis of Alzheimer's disease: cholinergic system linked to plaques, tangles and neuroinflammation. Curr Med Chem 13, 2179-2188 (2006).
Smith, W. W., Gorospe, M., Kusiak, J. W. Signaling mechanisms underlying Abeta toxicity: potential therapeutic targets for Alzheimer's disease. CNS Neurol Disord Drug Targets 5, 355-361 (2006).
Snyder, E. M., et al. Regulation of NMDA receptor trafficking by amyloid-beta. Nat Neurosci 8, 1051-1058 (2005).
Song, C., et al. Role of alpha7-nicotinic acetylcholine receptors in tetanic stimulation-induced gamma oscillations in rat hippocampal slices. Neuropharmacology 48, 869-880 (2005).
Sotthibundhu, A., et al. Beta-amyloid(1-42) induces neuronal death through the p75 neurotrophin receptor. J Neurosci 28, 3941-3946 (2008).
Spencer, J. P., et al. Transgenic mice over-expressing human beta-amyloid have functional nicotinic alpha 7 receptors. Neuroscience 137, 795-805 (2006).
Taly, A., et al. Nicotinic receptors: allosteric transitions and therapeutic targets in the nervous system. Nat Rev Drug Discov 8, 733-750 (2009).
Teaktong, T., et al. Nicotinic acetylcholine receptor immunohistochemistry in Alzheimer's disease and dementia with Lewy bodies: differential neuronal and astroglial pathology. J Neurol Sci 225, 39-49 (2004).
Thinschmidt J. S., et al. Medial septal/diagonal band cells express multiple functional nicotinic receptor subtypes that are correlated with firing frequency. Neurosci Lett 389, 163-168 (2005).
Valincius, G., et al. Soluble amyloid beta-oligomers affect dielectric membrane properties by bilayer insertion and domain formation: implications for cell toxicity. Biophys J 95, 4845-4861 (2008).
Viola, K. L., et al. Why Alzheimer's is a disease of memory: the attack on synapses by A beta oligomers (ADDLs). J Nutr Health Aging 12, 51S-57S (2008).
Vitolo, O. V., et al. Amyloid beta-peptide inhibition of the PKA/CREB pathway and long-term potentiation: reversibility by drugs that enhance cAMP signaling. Proc Natl Acad Sci U S A 99, 13217-13221 (2002).
Walsh, D. M., Selkoe, D. J. Deciphering the molecular basis of memory failure in Alzheimer's disease. Neuron 44, 181-193 (2004).
Wang, H. Y., et al. beta-Amyloid(1-42) binds to alpha7 nicotinic acetylcholine receptor with high affinity. Implications for Alzheimer's disease pathology. J Biol Chem 275, 5626-5632 (2000a).
Wang, H. Y., et al. Amyloid peptide Abeta(1-42) binds selectively and with picomolar affinity to alpha7 nicotinic acetylcholine receptors. J Neurochem 75, 1155-1161 (2000b).
Wang H. Y., et al. beta-Amyloid(1-42) binds to alpha7 nicotinic acetylcholine receptor with high affinity. Implications for Alzheimer's disease pathology. J Biol Chem 275, 5626-5632 (2000c).
Wang, H. Y., et al. Dissociating beta-amyloid from alpha 7 nicotinic acetylcholine receptor by a novel therapeutic agent, S 24795, normalizes alpha 7 nicotinic acetylcholine and NMDA receptor function in Alzheimer's disease brain. J Neurosci 29, 10961-10973 (2009a).
Wang, N. C., et al. EEG stages predict treatment response in experimental status epilepticus. Epilepsia 50, 949-952 (2009b).
Westmark, C. J., et al. Seizure Susceptibility and Mortality in Mice that Over-Express Amyloid Precursor Protein. Int J Clin Exp Pathol 1, 157-168 (2008).
Whiteaker, P., et al. Discovery, synthesis, and structure activity of a highly selective alpha7 nicotinic acetylcholine receptor antagonist. Biochemistry 46, 6628-6638 (2007).
Whitehouse, P. J., Kalaria, R. N. Nicotinic receptors and neurodegenerative dementing diseases: basic research and clinical implications. Alzheimer Dis Assoc Disord 9 Suppl 2, 3-5 (1995).
Whiting P. J., et al. Functional acetylcholine receptor in PC12 cells reacts with a monoclonal antibody to brain nicotinic receptors. Nature 327, 515-518 (1987).
Williams, J. H., Kauer, J. A. Properties of carbachol-induced oscillatory activity in rat hippocampus. J Neurophysiol 78, 2631-2640 (1997).
Wooltorton, J. R., et al. Differential desensitization and distribution of nicotinic acetylcholine receptor subtypes in midbrain dopamine areas. J Neurosci 23, 3176-3185 (2003).
Wu J., Chan P., et al. 1-Methyl-4-phenylpridinium (MPP+)-induced functional run-down of GABA(A) receptor-mediated currents in acutely dissociated dopaminergic neurons. J Neurochem 83 87-99 (2002).
Wu J., et al. beta-Amyloid directly inhibits human alpha4beta2-nicotinic acetylcholine receptors heterologously expressed in human SH-EP1 cells. J Biol Chem 279, 37842-37851 (2004a).
Wu J., et al. Electrophysiological, pharmacological, and molecular evidence for alpha7-nicotinic acetylcholine receptors in rat midbrain dopamine neurons. J Pharmacol Exp Ther 311, 80-91 (2004b).
Wu, J., et al. Roles of nicotinic acetylcholine receptor beta subunits in function of human alpha4-containing nicotinic receptors. J Physiol 576, 103-118 (2006).
Wu, M. N., et al. Involvement of nicotinic acetylcholine receptors in amyloid beta-fragment-induced intracellular Ca(2+) elevation in cultured rat cortical neurons. Sheng Li Xue Bao 61, 517-525 (2009).
Xiu, J., et al. Expression of nicotinic receptors on primary cultures of rat astrocytes and up-regulation of the alpha7, alpha4 and beta2 subunits in response to nanomolar concentrations of the beta-amyloid peptide(1-42). Neurochem Int 47, 281-290 (2005).
Yan Z., Feng J. Alzheimer's disease: interactions between cholinergic functions and beta-amyloid. Curr Alzheimer Res 1, 241-248 (2004).
Yang, D. S., et al. Neuronal apoptosis and autophagy cross talk in aging PS/APP mice, a model of Alzheimer's disease. Am J Pathol 173, 665-681 (2008a).
Yang, K., et al. Distinctive nicotinic acetylcholine receptor functional phenotypes of rat ventral tegmental area dopaminergic neurons. J Physiol 587, 345-361 (2009).
Yang, T., et al. Small molecule, non-peptide p75 ligands inhibit Abeta-induced neurodegeneration and synaptic impairment. PLoS One 3, e3604 (2008b).
Yu C. R. Role L. W. Functional contribution of the alpha5 subunit to neuronal nicotinic channels expressed by chick sympathetic ganglion neurones. J Physiol 509 (Pt 3), 667-681 (1998).
Yu, W. F., et al. High selective expression of alpha7 nicotinic receptors on astrocytes in the brains of patients with sporadic Alzheimer's disease and patients carrying Swedish APP 670/671 mutation: a possible association with neuritic plaques. Exp Neurol 192, 215-225 (2005).
Zhao, L., et al. Functional properties of homomeric, human alpha 7-nicotinic acetylcholine receptors heterologously expressed in the SH-EP1 human epithelial cell line. J Pharmacol Exp Ther 305, 1132-1141 (2003).

1. A method of treating a neurodegenerative disorder in an individual, comprising:
providing a composition capable of inhibiting dysfunctional signaling of α7 nicotinic acetylcholine receptors (nAChRs); and
administering a therapeutically effective amount of the composition to inhibit dysfunctional signaling of α7 nAChRs to treat the neurodegenerative disorder.

2. The method of claim 1, wherein the α7 nAChRs comprise heteromeric α7β2 nAChRs.

3. The method of claim 1, wherein the composition capable of inhibiting dysfunctional signaling of α7 nAChRs comprises comprises a β2 nAChR antagonist.

4. The method of claim 1, wherein the composition capable of inhibiting dysfunctional signaling of α7 nAChRs comprises an α7 nAChR antagonist.

5. The method of claim 1, wherein the neurodegenerative disorder comprises Alzheimer's Disease, dementia, Parkinson's Disease and/or epilepsy.

6. The method of claim 1, wherein the neurodegenerative disorder comprises an early stage form of Alzheimer's Disease.

7. The method of claim 1, wherein the composition capable of inhibiting dysfunctional signaling of α7 nAChRs comprises a compound comprising kynurenic acid (KYNA), methyllycaconitine (MLA), α-bungarotoxin (BGT), cholinesterase inhibitor, memantine, and/or α-conotoxin, or a pharmaceutical equivalent, derivative, analog and/or salt thereof.

8. The method of claim 1, wherein inhibiting the dysfunctional signaling of α7 nAChRs comprises restoring function of α7β2 nAChRs.

9. The method of claim 1, wherein inhibiting the dysfunctional signaling of α7 nAChRs comprises protecting α7β2 nAChRs from amyloid β (Aβ) effects.

10. The method of claim 1, wherein inhibiting the dysfunctional signaling of α7 nAChRs comprises a reduction in neuronal hyperexcitation.

11. The method of claim 1, wherein the individual is a human.

12. The method of claim 1, wherein the individual is a rodent.

13. The method of claim 1, wherein the dysfunctional signaling of α7 nAChRs occurs in the brain medial septum and/or diagonal band in the individual.

14. The method of claim 1, wherein the dysfunctional signaling of α7 nAChRs occurs in the hippocampus in the individual.

15. A method of diagnosing a neurodegenerative disorder in an individual, comprising:
obtaining a sample from the individual;
assaying the sample to determine the presence or absence of dysfunctional signaling of α7 nicotinic acetylcholine receptors (nAChRs) in the individual; and
diagnosing the neurodegenerative disorder based on the presence of dysfunctional signaling of α7 nAChRs in the individual.

16. The method of claim 15, wherein the α7 nAChRs comprise heteromeric α7β2 nAChRs.

17. The method of claim 15 wherein the individual is a human.

18. The method of claim 15 wherein the individual is a rodent.

19. The method of claim 15 wherein the neurodegenerative disorder comprises Alzheimer's Disease, dementia, Parkinson's Disease and/or epilepsy.

20. The method of claim 15 wherein the dysfunctional signaling of α7 nAChRs occurs in the brain medial septum and/or diagonal band in the individual.

21. The method of claim 15 wherein the dysfunctional signaling of α7 nAChRs occurs in the hippocampus in the individual.

22. The method of claim 15 wherein the neurodegenerative disorder is non-responsive to treatment with galantamine, or a pharmaceutical equivalent, derivative, analog and/or salt thereof.

23. The method of claim 15, wherein prior to obtaining the sample the individual is suspected of having a neurodegenerative disorder.

24. The method of claim 15, wherein prior to obtaining the sample the individual demonstrates susceptibility to seizures.

25. The method of claim 15, wherein prior to obtaining the sample the individual demonstrates abnormal θ oscillations.

26. A method of prognosing the onset of Alzheimer's Disease and/or dementia in an individual, comprising:
obtaining a sample from the individual;
assaying the sample to determine the presence or absence of dysfunctional signaling of α7 nicotinic acetylcholine receptors (nAChRs) in the individual; and
prognosing the onset of Alzheimer's Disease and/or dementia based on the presence of dysfunctional signaling of α7 nAChRs in the individual.

27. The method of claim 26, wherein the α7 nAChRs comprise heteromeric α7β2 nAChRs.

28. The method of claim 26 wherein the dysfunctional signaling of α7 nAChRs occurs in the brain medial septum and/or diagonal band in the individual.

29. The method of claim 26 wherein the dysfunctional signaling of α7 nAChRs occurs in the hippocampus in the individual.

30. A method of diagnosing an increased likelihood of an individual developing a neurodegenerative disorder relative to a normal subject, comprising:
obtaining a sample from the individual;
assaying the sample to determine the presence or absence of dysfunctional signaling of α7 nicotinic acetylcholine receptors (nAChRs) in the individual; and
diagnosing an increased likelihood of developing the neurodegenerative disorder relative to the normal subject based on the presence of dysfunctional signaling of α7 nAChRs in the individual.

31. The method of claim 30, wherein the α7 nAChRs comprise heteromeric α7β2 nAChRs.

32. The method of claim 30, wherein the neurodegenerative disorder comprises Alzheimer's Disease, dementia, Parkinson's Disease and/or epilepsy.

33. The method of claim 30, wherein prior to obtaining the sample the individual is suspected of having a neurodegenerative disorder.

34. The method of claim 30, wherein prior to obtaining the sample the individual demonstrates susceptibility to seizures.

35. A kit, comprising:
a quantity of a composition capable of detecting the presence or absence of dysfunctional signaling and/or expression of α7 nicotinic acetylcholine receptors (nAChRs); and
instructions for obtaining a sample from an individual, assaying the sample to determine the presence or absence of dysfunctional signaling and/or expression of nAChRs in the individual, and diagnosing an increased likelihood of developing a neurodegenerative disorder relative to the normal subject based on the presence of dysfunctional signaling and/or expression of α7 nAChRs in the individual.

36. The kit of claim 35, wherein the α7 nAChRs comprise heteromeric α7β2 nAChRs.

37. The kit of claim 35, wherein the neurodegenerative disorder comprises Alzheimer's Disease, dementia, Parkinson's Disease and/or epilepsy.

38. The kit of claim 35, wherein the kit is disposable.


SEQUENCE LISTING (8 sequences)

SEQ ID NO: 1 - DNA, 45 bp, Homo sapiens
  misc_feature (22)..(22): n is a, c, g, or t
atttaggtgacactatagaagnggatcatcgtgggcctctcagtg

SEQ ID NO: 2 - DNA, 44 bp, Homo sapiens
taatacgactcactatagggagagttggcgatgtagcggacctc

SEQ ID NO: 3 - DNA, 47 bp, Homo sapiens
  misc_feature (22)..(22): n is a, c, g, or t
atttaggtgacactatagaagngtcacggtgttcctgctgctcatct

SEQ ID NO: 4 - DNA, 47 bp, Homo sapiens
taatacgactcactatagggagatcctccctcacactctggtcatca

SEQ ID NO: 5 - DNA, 32 bp, Homo sapiens
cggagcatttgaactctgagcagtggggtcgc

SEQ ID NO: 6 - DNA, 24 bp, Homo sapiens
ctcgctgacacaagggctgcggac

SEQ ID NO: 7 - DNA, 26 bp, Homo sapiens
cactacgtctgaacgtcgaaaacccg

SEQ ID NO: 8 - DNA, 26 bp, Homo sapiens
cgggcaaataatatcggtggccgtgg